SEQUENCE LISTING (1 sequence)

SEQ ID NO: 1 - DNA, 20 bp, Baceteriophage T3
aattaaccctcactaaaggg

(19) United States
(12) Patent Application Publication
(10) Pub. No.: US 2016/0355869 A1
(43) Pub. Date: Dec. 8, 2016

(54) BIOSENSORS INCLUDING METALLIC NANOCAVITIES

(71) Applicant: University of Utah Research Foundation, Salt Lake City, UT (US)

(72) Inventors: Steven M. Blair, Salt Lake City, UT (US); Farhad Mahdavi, Salt Lake City, UT (US); Yongdong Liu, Salt Lake City, UT (US); James N. Herron, Salt Lake City, UT (US)

(73) Assignee: University of Utah Research Foundation, Salt Lake City, UT (US)

(21) Appl. No.: 14/595,100

(22) Filed: Jan. 12, 2015

Related U.S. Application Data
(63) Continuation-in-part of application No. 11/497,581, filed on Aug. 2, 2006, now Pat. No. 9,012,207.
Application No. 11/497,581 is a continuation-in-part of application No. 12/793,883, filed on Jun. 4, 2010, now abandoned.
(60) Provisional application No. 60/705,216, filed on Aug. 2, 2005.
(60) Provisional application No. 60/795,110, filed on Apr. 26, 2006.

(51) Int. Cl. C12Q 1/68; B01L 3/00; G01N 21/65; G01N 33/553; G01N 33/543

(52) U.S. Cl. CPC C12Q 1/682; C12Q 1/6837; C12Q 1/6825; G01N 33/553; G01N 33/54373; G01N 21/658; B01L 3/5085; B01L 2300/0896; B01L 2300/0887; B01L 2300/0819; B01L 2300/12; B01L 2300/168; B01L 2300/0858

(57) ABSTRACT

Devices and methods relating to biological assays are provided. In one exemplary aspect, a detection-enhancement element for a biological assay can include a substrate, a metallic layer on at least one surface of the substrate and including at least one nanocavity, a transparent film positioned between the substrate and the metallic layer; and capture molecules within the at least one nanocavity. The nanocavities are configured to enhance signals that are representative of the presence or amount of one or more analytes in a sample or sample solution, and may be configured to enhance the signal by a factor of about two or more or by a factor of about three or more. Such signal enhancement may be achieved with nanocavities that are organized in an array, randomly positioned nanocavities, or nanocavities that are surrounded by increased surface area features, such as corrugation or patterning, or nanocavities that have quadrilateral or triangular shapes with tailored edge lengths, or with a plurality of nanoparticles. Methods for fabricating biomolecular substrates and assay techniques in which such biomolecular substrates are used are also disclosed.

### PRIORITY DATA

This application is a continuation-in-part of U.S. patent application Ser. No. 11/497,581, filed on Aug. 2, 2006, which claims the benefit of U.S. Provisional Patent Application Ser. No. 60/705,216, filed on Aug. 2, 2005, and which also claims the benefit of U.S. Provisional Patent Application Ser. No. 60/795,110, filed on Apr. 26, 2006, both of which are incorporated herein by reference in their entireties. This application is also a continuation-in-part of U.S. patent application Ser. No. 12/793,883, filed on Jun. 4, 2010, which is incorporated herein by reference.

### GOVERNMENT INTEREST

This invention was made with government support under R21 EB000481 awarded by National Institutes of Health, and ECS0134548, ECS0622225 and ECS0637121 awarded by National Science Foundation. The government has certain rights in the invention.

### FIELD OF THE INVENTION

The invention relates generally to evanescent wave-type biosensors, or biomolecular assays. More specifically, the invention relates to biosensors including substrates with metallic films on one or more surfaces thereof and, in particular, to biosensors with metallic films that include nanocavities with shapes that are configured to optimize the amplification of signals indicative of the presence or amount of one or more analytes present in a sample.

### BACKGROUND

Plasmonics is the study of phenomena related to the interaction of electromagnetic radiation with an electron gas (or plasma) at a metal surface. Aside from the now-common surface plasmon resonance (SPR)-based sensors, plasmonics has been applied to molecular detection applications by attaching metallic nanoparticles to molecules for use as light scattering labels in biosensing. Nanostructured metallic surfaces have also been studied extensively for surface-enhanced fluorescence and Raman scattering (SERS). One of the major drawbacks of these surface-enhanced techniques is that the nanostructure is disordered (but sometimes with fractal order) such that the fluorescence or Raman enhancement factors are spatially-varying, as evidenced by “hot-spots” on the surface. The hot-spot effect may render these techniques unsuitable for quantitative assays, especially in an array format, as the average enhancement over a defined sensing zone may not be very high, and the enhancement from zone to zone may vary. As a result, there have been efforts in which molecules are attached to lithographically defined arrays of metallic nanoparticles. With these architectures, uniformity in nanoparticle size, shape, and spacing result in well-defined enhancement in terms of magnitude and spatial location. However, these techniques do not provide complete isolation from background produced by unbound species, as uniform illumination can excite fluorescence from molecules located between nanoparticles, which produce background signals at the detector.

An important recent advance is the demonstration of extraordinary light transmission through a periodic array of subwavelength metallic apertures or nanocavities, where, in the absence of the nanocavities, no light passes through the metal film. Even though this has been quite an active area of research, some disagreement about the origin of the transmission enhancement still exists. However, it is generally believed that the periodic array of nanocavities acts as a two-dimensional diffraction grating, which, at specific incidence angles, allows light to couple from free space into surface plasmon polariton (SPP) Bloch modes on either metal interface. These SPP modes can constructively interfere within the nanocavities, resulting in intensity enhancement and, therefore, greater transmission. It has been demonstrated experimentally, using fluorophores as local intensity probes, that light is indeed localized within the nanocavities and that enhanced fluorescence transduction can be performed.

### BRIEF DESCRIPTION OF THE DRAWINGS

For a fuller understanding of the nature and advantage of the present disclosure, reference is being made to the following detailed description of various embodiments and in connection with the accompanying drawings, in which:

FIG. 1a schematically depicts a plan view of a periodic array of nanocavities according to an aspect of the present disclosure.

FIG. 1b schematically depicts a plan view of a “bullseye” structure of a single nanocavity surrounded by an annular, corrugated grating according to an aspect of the present disclosure.

FIG. 1c schematically depicts a plan view of a periodic array of nanoparticles according to an aspect of the present disclosure.

FIG. 1d schematically depicts a cross section view of a periodic array of nanocavities according to an aspect of the present disclosure.

FIG. 1e schematically depicts a cross section view of a “bullseye” structure of a single nanocavity surrounded by an annular corrugated grating according to an aspect of the present disclosure.

FIG. 1f schematically depicts a cross section view of a periodic array of nanoparticles according to an aspect of the present disclosure.

FIG. 2a schematically depicts a plan view of a random nanocavity array according to an aspect of the present disclosure.

FIG. 2b schematically depicts a plan view of a periodic nanocavity array according to an aspect of the present disclosure.

FIG. 2c schematically depicts a side view of the periodic nanocavity array of FIG. 2b according to an aspect of the present disclosure.

FIG. 3a schematically depicts a hybridization array according to an aspect of the present disclosure.

FIG. 3b schematically depicts an individual hybridization zone according to an aspect of the present disclosure, where each hybridization zone includes an array of metallic nanocavities.

FIG. 3c schematically depicts a cross section view of a nanocavity, where the probe molecules are selectively attached to the bottom of the nanocavity according to an aspect of the present disclosure.

FIG. 3d schematically depicts a cross section view of a nanocavity, where the probe molecules are selectively attached to the sides of the nanocavity according to an aspect of the present disclosure.

FIG. 3e schematically depicts a cross section view of an array of nanocavities where metal surfaces are passivated by depositing a thin layer of SiO2 according to an aspect of the present disclosure.

FIG. 4 depicts an example of microfluidic channels that may be used in a real-time hybridization experiment, according to one aspect of the present disclosure.

FIG. 5 is a graph that illustrates real-time hybridization between T3 in solution and anti-T3 immobilized within the nanocavities, according to one aspect of the present disclosure.

FIG. 6 schematically depicts the geometry associated with a two compartment model that simulates binding between capture molecules and target molecules, according to one aspect of the present disclosure.

FIG. 7 is a model association/dissociation curve, according to one aspect of the present disclosure.

FIG. 8 illustrates the patterning of individual detection zones of a 3×3 hybridization array for validation studies, according to one aspect of the present disclosure.

FIG. 9 is a scanning electron microscopy (SEM) image of a square lattice periodic nanoaperture array, according to one aspect of the present disclosure.

FIG. 10 is an SEM image of a metallic nano particle array fabricated through e-beam lithography with a lift-off process, according to one aspect of the present disclosure.

FIG. 11a is a cross-sectional side schematic view of a light enhancement or detection enhancement device for a biological assay, according to one aspect of the present disclosure.

FIG. 11b is a schematic view of an experimental configuration, according to one aspect of the present disclosure.

FIG. 12a is a graph of count rate per molecule versus the excitation power within a single 120 nm aperture with different adhesion layers, according to one aspect of the present disclosure. Markers are experimental data, solid lines are numerical fits. Fitting parameters are summarized in Table 1.

FIG. 12b is a graph of fluorescence rate enhancement in the regime below saturation deduced from the numerical fits in FIG. 12a, according to one aspect of the present disclosure.

FIG. 13a is a graph of normalized fluorescence decay traces measured in open solution (black dots) and in single 120 nm apertures with 10 nm Ti or TiO₂ adhesion layer, according to one aspect of the present disclosure. Dots are experimental data, lines are numerical fits. The other adhesion layers used in this study resulted in traces almost identical to the one of the TiO₂ case; they are therefore not represented here to maintain clarity.

FIG. 13b is a graph of fluorescence lifetime reduction as compared to molecules in open solution for the different adhesion layers, according to one aspect of the present disclosure.

FIG. 14a is a graph of contributions of excitation to the fluorescence enhancement found for different adhesion layers, according to one aspect of the present disclosure. Bars are experimental data, empty circles are for numerical computations.

FIG. 14b is a graph of Contributions of emission gains to the fluorescence enhancement found for different adhesion layers, according to one aspect of the present disclosure. Bars are experimental data, empty circles are for numerical computations.

### DETAILED DESCRIPTION

Before the present disclosure is described herein, it is to be understood that this disclosure is not limited to the particular structures, process steps, or materials disclosed herein, but is extended to equivalents thereof as would be recognized by those ordinarily skilled in the relevant arts. It should also be understood that terminology employed herein is used for the purpose of describing particular embodiments only and is not intended to be limiting.

### DEFINITIONS

The following terminology will be used in accordance with the definitions set forth below.

It should be noted that, as used in this specification and the appended claims, the singular forms “a,” and, “the” include plural referents unless the context clearly dictates otherwise. Thus, for example, reference to “a nanocavity” includes one or more of such nanocavities and reference to “the layer” includes reference to one or more of such layers.

In this application, “comprises,” “comprising,” “containing” and “having” and the like can have the meaning ascribed to them in U.S. Patent law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. Patent law. “Consisting essentially of” or “consists essentially of” have the meaning generally ascribed to them by U.S. Patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the composition's nature or characteristics would be permissible if present under the “consisting essentially of” language, even though not expressly recited in a list of items following such terminology. When using an open ended term, like “comprising” or “including,” it is understood that direct support should be afforded also to “consisting essentially of” language as well as “consisting of” language as if stated explicitly, and vice versa. Further, it is to be understood that the listing of components, species, or the like in a group is done for the sake of convenience and that such groups should be interpreted not only in their entirety, but also as though each individual member of the group has been articulated separately and individually without the other members of the group unless the context dictates otherwise. This is true of groups contained both in the specification and claims of this application. Additionally, no individual member of a group should be construed as a de facto equivalent of any other member of the same group solely based on their presentation in a common group without indications to the contrary.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a numerical range endpoint by providing that a given value may be “a little above” or “a little below” the endpoint.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually.

This same principle applies to ranges reciting only one numerical value as a minimum or a maximum. Furthermore, such an interpretation should apply regardless of the breadth of the range or the characteristics being described.

### The Disclosure

A biomolecular assay includes a substrate with a metallic film, or layer, on at least one surface thereof. The metallic film includes nanocavities. The nanocavities are configured to enhance signals that are representative of the presence or amount of one or more analytes (e.g., proteins or peptides, nucleic acids, small molecule ligands, ions, etc.) in a sample or sample solution. Such a biomolecular assay may be used for a variety of purposes, including, without limitations, receptor-ligand binding, drug screening, real-time nucleic acid hybridization, clinical diagnostics, etc.

A biomolecular assay may be fabricated by forming a substrate, or support, from a suitable material (e.g., glass, quartz, another optically suitable (e.g., transparent) inorganic material, an optical plastic, a combination of any of the foregoing (as is the case in so-called “thin-film” waveguides, which include multiple layers), etc.). A metallic film, or layer, is applied to at least one surface of the substrate (e.g., by deposition techniques, lamination processes, etc.). By way of nonlimiting example, the metallic film may have a thickness of about 100 nm.

Nanocavities are formed in the metallic film by suitable processes (e.g., mask and lift-off processes (such as those used in semiconductor device fabrication), mask and etch processes (such as those used in semiconductor device fabrication), with a laser, etc.). The nanocavities may extend completely through the metallic film, with the underlying substrate being exposed therethrough. A lateral dimension (e.g., diameter) of each nanocavity may be about the same as the thickness of the metallic layer, although lateral nanocavity dimensions may differ from the thickness of the metallic layer.

Nanocavities of virtually any shape may be formed. Examples of nanocavity shapes include, but are not limited to, round (e.g., circular, oval, elliptical, egg-shaped, etc.), quadrilateral (e.g., square, rectangular, parallelogram, trapezoidal, etc.), triangular, and other polygonal shapes. The nanocavities that are formed in a metallic film may all have substantially the same shapes and dimensions, or a variety of shapes and/or dimensions of nanocavities may be included in the metallic film of a biomolecular assay that incorporates teachings of the invention.

The nanocavities may be arranged in such a way that facilitates the coupling of incident light into surface modes, or waves, on the metallic film, which surface modes can constructively interfere within the nanocavities. For example, when incident light is to be directed from the substrate, or back side of the biomolecular assay, and fluorescence is to be detected at a location adjacent to the opposite, top surface of the biomolecular assay (i.e., the surface by which the metallic film is carried), the metallic film prevents excitation of fluorophores in the bulk solution, which is located over the metallic substrate. As another example, when incident light is directed toward the biomolecular assay from a location over the metallic film and detection occurs at a location adjacent to the back side of the substrate, although marker molecules that remain within solution may undergo a change in state (e.g., fluorescence by fluorescent marker molecules), the marker molecules that remain in solution over the metallic film remain substantially undetected. This is because light emitted from a location above the metallic film does not pass through the metallic film and since the size of each nanocavity may be too small for fluorescent light emitted from locations over the surface of the metallic film to pass therethrough. Fluorescent light generated within the nanocavities does exit the nanocavities, however, and is enhanced by the materials from which the nanocavities are formed, as well as by the configurations and dimensions of the nanocavities.

In specific applications, however, fluorescence signals originating from fluorescent species lying outside of the cavity may be a concern. For example, these signals may increase background or noise of an assay and thus compromise the sensitivity and/or precision of the assay. Partial or complete isolation of fluorescence signals originating from fluorescent species lying outside of the cavity can be obtained by either narrowing the fluorescence collection angle or by passivating the surfaces of the metallic film.

The shapes of the nanocavities may be configured to optimize signal amplification. It has been discovered that nanocavities of a variety of shapes, including circular, square, and triangular, provide a good degree of radiative, or signal, enhancement, depending upon the dimensions (e.g., diameters of circular nanocavities, edge lengths of square and triangular nanocavities, etc.) of the nanocavities. Square nanocavities may provide better signal enhancement than circular nanocavities, while triangular nanocavities may provide even greater signal enhancement. Without limiting the scope of the invention, circular nanocavities formed in a gold film having a thickness of about 100 nm may enhance a signal by up to about 1.8 times (for nanocavities having diameters of about 160 nm), square nanocavities formed in a gold film having a thickness of about 100 nm may enhance a signal by up to about 2.1 or 2.2 times (for nanocavities with edges of about 125 nm in length and 20 nm radius corners), and equilateral triangular cavities formed in a gold film having a thickness of about 100 nm may enhance a signal by up to about 3 times (for nanocavities with edges that are about 175 nm long an that have 20 nm radius corners). It is believed that even greater radiative, or signal, enhancements may be achieved by further tailoring the shapes or dimensions of nanocavities.

Optionally, a biomolecular substrate according to the invention may include one or more transparent films positioned between the substrate and the metallic film or over the metallic film for directing incident light to the nanocavities. Nanocavities may extend into or through such transparent films.

Surfaces of the biomolecular substrate may be passivated to prevent capture molecules (e.g., bait molecules) from adhering, or being immobilized, to undesired locations thereof. The surfaces of the metallic film may be passivated, for example, with polyethylene glycol (PEG)-thiol, another metal (e.g., gold)-selective thiol molecule, or any other material that prevents capture molecules from being immobilized to the metallic film, or reduce immobilization of capture molecules to the metallic film. Thus, the capture molecules are instead immobilized to the surface of the substrate exposed to and located within or adjacent to the nanocavities. Alternatively, the exposed surfaces of the substrate may be passivated (e.g., with PEG silane) to prevent capture molecules from adhering to the substrate and, rather, causing the capture molecules to be immobilized only to the metallic surfaces. As another alternative, a major surface of the metallic film may be covered with a coating film (e.g., another transparent film), and the exposed surfaces of the coating film, as well as surfaces of the substrate that are exposed through the coating film and the metallic film, may be passivated, causing capture molecules to adhere only to the unpassivated exposed edges of the metallic film, which form part of the surface of each nanocavity.

The biomolecular substrate may be configured in such a way that surface modes (e.g., surface plasmons, which generate an evanescent field) may be generated at the surface of the metallic film or at the portions of the surface of the substrate that are exposed by the nanocavities. These surface modes may provide enhanced excitation of marker molecules on analyte that has been bound to capture molecules within the nanocavities, particularly surface modes that constructively interfere with one another.

Capture molecules are introduced into the nanocavities and immobilized to surfaces of the nanocavities, the substrate, or both, as known in the art. The capture molecules are specific for one or more analytes of interest.

Increased surface area structures, such as corrugated patterning having a “bullseye” configuration, other patterns, or the like may be formed around each nanocavity.

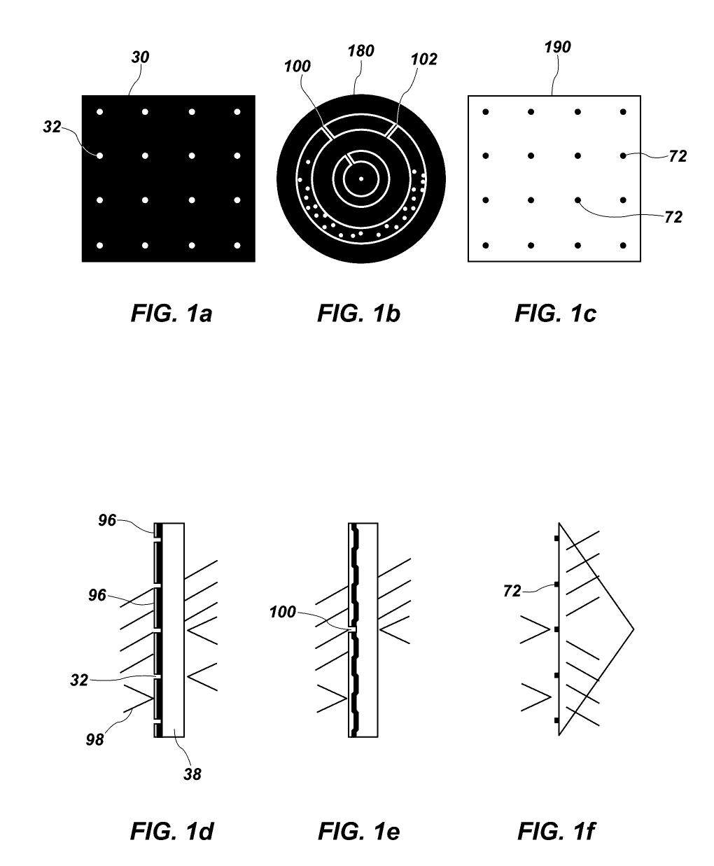

Nanocavities may be arranged in nanostructure architectures such as a periodic array of nanocavities, a random array of nanocavities, and “bullseye” structure of single nanocavity surrounded by an annular, corrugated grating. Several embodiments of nanostructure architectures are illustrated in FIGS. 1a-f as described herein.

A periodic array of metallic nanoparticles can also provide enhanced signals, since, in essence, metallic nanoparticles merge to form a periodic hole array. An example of a nanoparticle array is illustrated in FIG. 1c as described herein.

Such a biomolecular assay may include a sequential delivery system, in which a sample flows into the nanocavities in sequence. Sequential delivery systems are useful for detecting small concentrations of analyte, as well as with samples having small volumes. Of course, other types of delivery systems, including delivery systems with sections through which portions of a sample may flow in parallel, are also within the scope of the invention.

Once a sample has traveled through the delivery system, it may be recycled through the system one or more times. Recycling may be effected in a loop, in which the sample travels through the system in the same direction each time, or may be effected by reversing the direction in which the sample flows through the delivery system. Recycling may be useful for optimizing detection of low analyte concentrations in a sample or for detecting analyte in samples having small volumes.

Sample flow through a sequential or any other configuration of delivery system may be effected by mechanisms that are fabricated on or assembled with the biomolecular assay. Examples of flow facilitators include, but are not limited to, peristaltic pumps, positive pressure systems, and negative pressure systems.

Mixing structures can be utilized, such as those disclosed in U.S. patent application Ser. No. 10/350,361, filed on Jan. 23, 2003, the disclosure of which is hereby incorporated herein in its entirety. Such mixing structures may also be included in a sample delivery system of a biomolecular assay that incorporates or may use the invention to advantage. In addition or as an alternative to being located at reaction sites of the biomolecular assay, mixing structures may be positioned along other locations of the sample delivery system, including between reaction sites and at ends of the system, where they may increase the homogeneity of a sample prior to recirculating the sample through the delivery system.

The use of a sequential delivery system, sample recirculation, sample flow facilitation, sample mixing, or any combination thereof may reduce the amount of reagent required for detecting an analyte in a sample.

As a nonlimiting example, the biomolecular assay may be used in an assay system or technique that employs fluorescence detection techniques. Such a system also includes a source of electromagnetic radiation and a detector. The source is configured to emit electromagnetic radiation of one or more wavelengths, or “incident light,” that excites fluorescent dye molecules that are to be used in the system, and oriented to direct the radiation onto the nanocavities or into the substrate. The incident light may be in the form of light transmitted from a source, an evanescent field generated as light is directed into and internally reflected within a substrate or transparent film that comprises a waveguide, or a combination thereof. Radiation can penetrate the nanocavities directly and, optionally, due to constructive interference that may occur because of the arrangement of the nanocavities, thereby exciting species within the nanocavities, or radiation may be internally reflected within the substrate, generating an evanescent field at one or more surfaces thereof. The incident light excites fluorescent dye molecules that are immobilized (directly or indirectly, depending upon the assay binding technique (e.g., a sandwich-type assay, a binding competition assay, etc.) employed relative to capture molecules within the nanocavities. Fluorescent dye molecules within the nanocavities are excited and, thus, emit electromagnetic radiation. The electromagnetic radiation is enhanced by the nanocavities and the metallic substrate. It is then detected by the detector. An aperture associated with the detector may tailor the angle of a collection cone of radiation emitted by the fluorescent dye molecules.

A biomolecular assay that incorporates teachings of the invention may be used with known mass detection processes.

As an example, a reference analyte of known concentration and analyte within a sample, which has an unknown concentration, may be labeled with different marker molecules (e.g., fluorescent molecules that emit different wavelengths, or colors, of light) and their binding to capture molecules that have been immobilized within the nanocavities compared to provide an indication of the amount of analyte in the sample. The affinities of the reference analyte and the sample analyte for the capture molecule, which may be known, may be the same or different.

Of course, systems and techniques that employ chemiluminescence, photoluminescence, electroluminescence, and other types (i.e., no fluorescent) of marker molecules (e.g., metallic markers, such as the gold markers used in Raman scattering techniques, etc.) are also within the scope of the invention.

Furthermore, it would be advantageous to bridge the gap between numerical modeling and experimental observations of the influence of adhesion layers in plasmonics. In addition, it has been recognized that it would be advantageous to develop a light enhancement device and/or detection enhancement device for a biological assay.

In another aspect, the invention provides a detection-enhancement device for biological assay including a metallic layer disposed over a substrate. An array of multiple nanocavities extend into the metallic layer. The nanocavities each have a bottom and a sidewall laterally circumscribing each nanocavity. Capture molecules are disposed within the nanocavities. The metallic layer has a thickness between 50 to 200 nanometers, and the nanocavities have a lateral dimension of 65 to 190 nanometers. An adhesion layer adheres the metallic layer to the substrate. A thickness of the adhesion layer and the diameter of the cavity have a ratio in the range of 1:4 to 1:100.

In addition, the invention provides a light enhancement device includes at least two layers disposed over a substrate, comprising at least a first layer and a second layer. The first layer is disposed closer to the substrate than the second layer. At least one nanocavity extends into the second layer. The first layer has a first layer thickness and a material, the second layer has a second layer thickness and a material, and the at least one cavity has a cavity diameter and a cavity shape adapted to enhance transmission of light through the at least one nanocavity. The thickness of the first layer and the diameter of the cavity have a ratio that is in the range of approximately 1:4 to 1:100.

In addition, the invention provides a light enhancement device includes at least two layers disposed over a substrate, comprising at least a first layer and a second layer. The first layer is disposed closer to the substrate than the second layer. An array of multiple nanocavities extends into the second layer. The first layer has a first layer thickness and a material, the second layer has a second layer thickness and a material, the at least one cavity has a cavity diameter and a cavity shape adapted to enhance transmission of light through the at least one nanocavity. The first layer causes an improvement in light transmission by a factor of at least 3.

Furthermore, the invention provides a detection-enhancement device for biological assay, comprising a metallic layer disposed over a substrate. An array of multiple nanocavities extends into the metallic layer. The nanocavities each have a bottom and a sidewall laterally circumscribing each nanocavity. Capture molecules are disposed within the nanocavities. The metallic layer has a thickness between 50 to 200 nanometers, and the nanocavities have a lateral dimension of 65 to 190 nanometers. An adhesion layer adheres the metallic layer to the substrate. A blocking layer is disposed over the metallic layer includes a material to resist light transmitting through the metallic layer.

As has been described, the invention includes biosensors comprising metallic nanocavities, thereby providing an apparatus, elements of an apparatus, and methods of fabrication and using such an apparatus. In the following description, reference is made to the accompanying drawings, which show, by way of illustration, several embodiments of the invention.

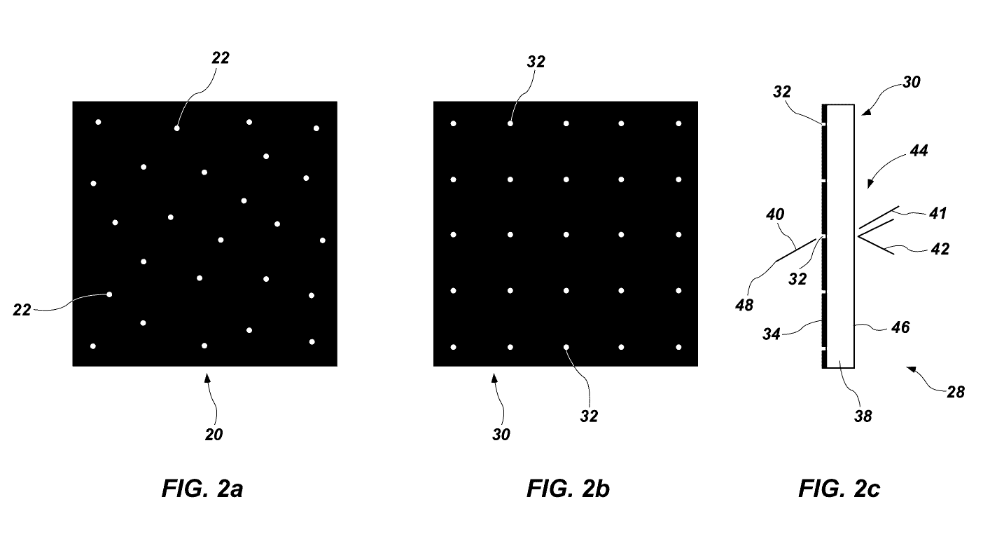

FIG. 2a-b illustrate a randomly arranged array 20 and a periodic arranged array 30 of nanocavities, 22 and 32, respectively. FIG. 2c illustrates a cross section 28 of FIG. 2b shown formed on a metallic layer 34 (indicated by solid black rectangles) on a surface 36 of a quartz substrate 38. FIG. 2c also shows optical paths 40, 41 and 42, and geometrical parameters d and A of the nanocavities, where d is the nanocavity diameter and A is the spacing of adjacent nanocavities. As illustrated in FIG. 2c, when incident light 40 is directed toward the nanocavities 32 from a location 48 over the metallic layer 34, enhanced fluorescence output of fluorescent molecules within the nanocavities 32 may be detected at a location 44 adjacent to a back side 46 of the substrate 38. For example, fluorescence output of fluorescent molecules within nanocavities may be read out using standard fluorescence scanners (i.e., normal incidence excitation, normal incidence fluorescence collection) from the backside 46 of the substrate. However, fluorescence output may also be detected in a reflection mode rather than a transmission mode as described herein.

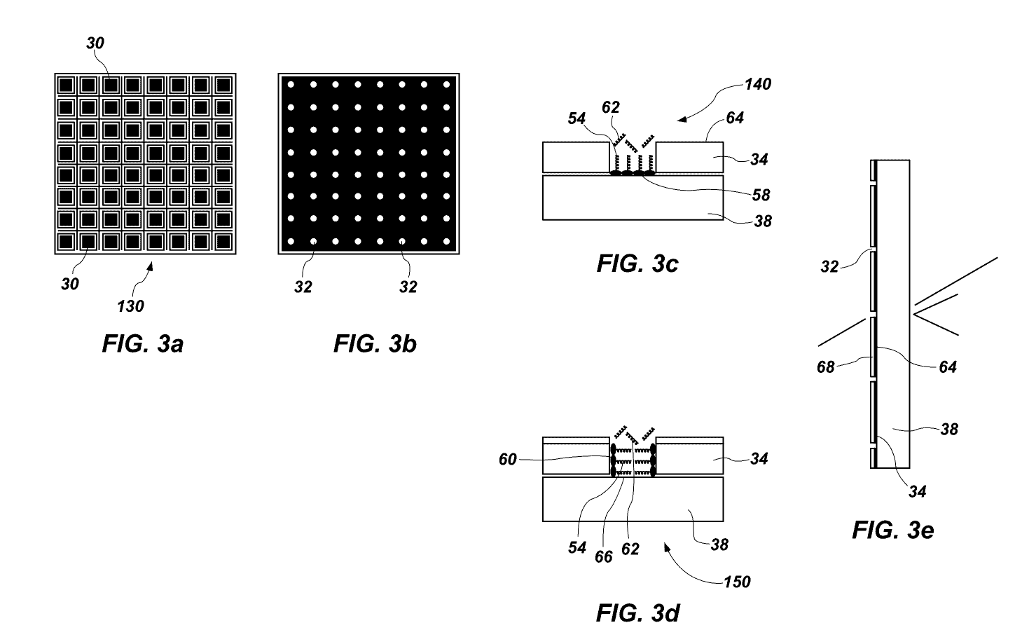

Target or capture molecules of an assay may reside on the sidewalls, the bottom surfaces, or both the sidewalls and the bottom surfaces of the nanocavities. Two nanocavity embodiments are illustrated in FIG. 3a-e: Type I (as shown in FIG. 3cc), where capture molecules 54 (e.g., probe oligonucleotides) are selectively attached to the bottoms 58 of the nanocavities, and Type II (as shown in FIG. 3d), where the probes 54 are selectively attached to the sidewalls 60 of the nanocavities. As shown in FIG. 3a, a hybridization array 130 comprises one or more sub-arrays or periodic array 30 of metallic nanocavities within which capture, or probe, oligonucleotides 54 are tethered directly to the nanocavities 32. One molecular species 62 (i.e., the target oligonucleotides or analyte, which is fluorescently labeled) specifically bind to the capture oligonucleotides 54 through hybridization. An optically-transduced, real-time signal in proportion to the number of bound target oligonucleotides are detected from the side of the sensor array opposite to the side on which the sample solution is introduced, thereby providing isolation from unbound species, which may represent a significant fraction of the detected signal in a washless assay. In this nanocavity array architecture, fluorescence from unbound species 98, as shown in FIG. 1a, do not penetrate the opaque metal except at the nanocavities. By measuring the hybridization kinetics in real-time, non-specific binding may be factored out as well. Study of fluorescence enhancements using the two embodiments also allows for independent and direct measurement of enhancement factors for molecules located at the bottom surfaces (Type I) and at the sidewalls (Type II).

However, fluorophores placed in close proximity to the exposed metal surface of a periodic nanocavity structure may couple to surface plasmons and emit from the back side of the substrate at specific angles. Isolation from this fluorescence signal may be obtained by either narrowing the collection angle to exclude this contribution or by passivating the surfaces of the metallic film.

To prevent capture molecules from binding to undesired locations of the nanocavities, the surfaces of the metallic film or the surface of the substrate may be passivated. For example, for Type I nanocavities (FIG. 3c), a surfaces 64 of the metallic film 34 may be passivated with polyethylene glycol (PEG)-thiol. Thus, the probe oligonucleotides (i.e., capture molecules) are selectively attached to the bottoms of the nanocavities 32. On the other hand, for Type II nanocavities (FIG. 3d), the exposed surface 66 of the substrate 38 may be passivated (e.g., with PEG silane) to prevent capture molecules 54 from adhering to the surface 66 of the substrate 38 and, rather, causing the capture molecules 54 to be selectively attached to the sidewalls 60 of the nanocavities. Alternatively, as shown in FIG. 3e, to facilitate coupling of the capture molecules 54 to the sidewalls 60 of the nanocavities, a thin (^{˜}20 nm) layer 68 of SiO₂ is deposited to cover the top metal surface 64. With this structure, the only exposed substrate surface is the inside walls of the nanocavities, to which selective derivatization of capture molecules is performed. A layer (e.g., ^{˜}5 nm) of Al or Cr, which is not shown in FIG. 3e, may be used to promote adhesion of the SiO₂ layer.

Surfaces of nanocavity substrates may also be modified for covalent or noncovalent immobilization of capture molecules. For example, SiO₂ and Si₃N₄ surfaces of nanocavity substrates may be modified with a reactive species, e.g., epoxysilane. After surface modification with a reactive species, coating of the capture molecules may be performed. For example, amine-modified nucleic acid probes may be spotted on epoxysilane-modified surfaces, and reactions of the amine groups and the epoxy groups cause the amine-modified nucleic acid probes covalently linked to the surface of the substrate.

Not wishing to be bound by theory, the surface plasmon modes at each of the metal interfaces in general have different propagation constants, such that usually only one of these modes (the one at the metal-air interface) plays a role in enhanced transmission. However, the modes at the two interfaces may be coupled together. Fluorescence excitation enhancement may be improved by surface plasmon cross-coupling. Surface plasmon cross-coupling may be achieved by controlling the refractive index at the substrate by depositing a thin film of silicon oxynitride (SiON) between the metallic film and quartz substrate. FIG. 1a shows a SiON layer 96 deposited on the metallic surface 34. For example, with SiON deposited by plasma-enhanced chemical vapor deposition, one can have continuous control over refractive index in the range 1.46 to 2.05 by adjusting the relative ratio of oxygen and nitrogen atoms. For Type II nanocavities as shown in FIG. 3d, the effects of the finite thickness of SiO₂ on the top surface should also be taken into account in the propagation constant of the surface plasmon wave at that interface.

Nanocavity geometry also has a strong influence on the emission properties of the fluors within a nanocavity, most likely based upon the nanocavity aspect ratio h/d, where h is the height or depth of the nanocavity. Geometric properties, such as spacing Λ and the incident angle of the incident light, may also be optimized to maximize surface plasmon coupling.

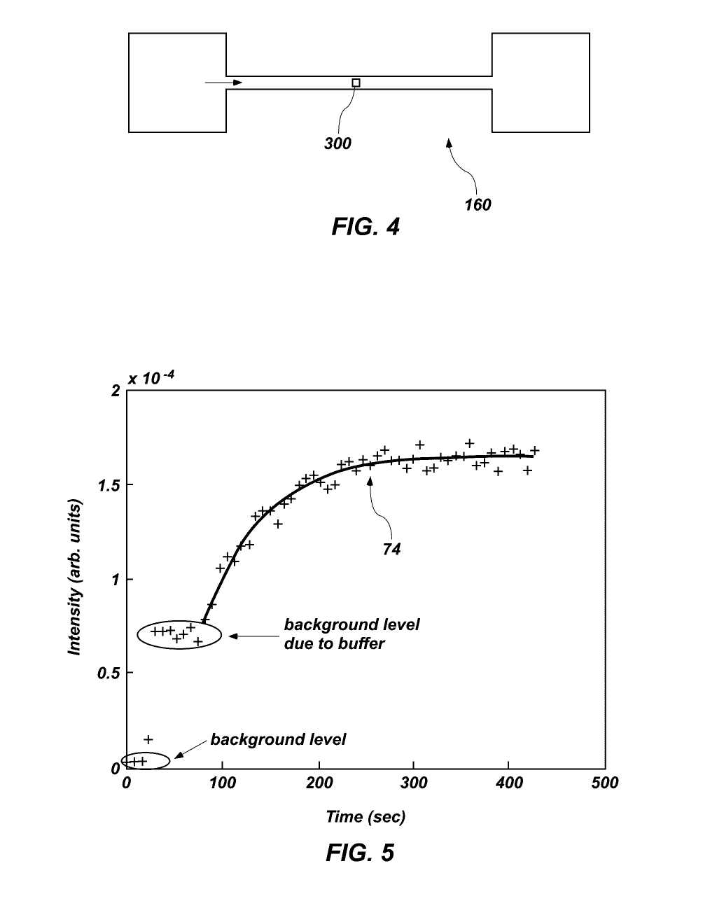

A sample may be delivered to the array of hybridization zones through a flow cell or a microfluidic channel. As shown in FIG. 4, a nanocavity array 300 is placed within a microfluidic channel 160. Sample flow is produced by a syringe pump with suitable a flow rate, e.g., of 0.3 mL/min. In order to avoid binding of target to the inside surface of the channels, the interior surface area of the channels may be passivated, e.g., with bovine serum albumin (BSA).

Sub-arrays of metallic nanostructures may provide highly sensitive, real-time detection. Three periodic metallic nanostructures are shown schematically in FIG. 1a-f. FIG. 1a-c illustrate a periodic array 30 of nanocavities, a so-called “bullseye” structure 180 and a modified periodic array 190 of nanoparticles. The structure 180 being of single nanocavity 100 surrounded by an annular, corrugated grating 102. Cross sections corresponding to the respective nanostructures, showing geometrical parameters and optical paths, are also respectively shown in FIG. 1d-f.

In the first two architectures (FIG. 1a-b), the nanocavities 32 serve as the binding and detection sites of a target entity, and in the third architecture (FIG. 1cc), the nanoparticles 72 serve as the binding and detection sites. Enhanced fluorescence transduction occurs through the optical excitation of molecules preferentially bound within metallic nanocavities or attached to metallic nanoparticles.

Not wishing to be bound by theory, enhanced fluorescence of the three nanostructures of FIG. 1a-c occurs through two mechanisms—increased fluor excitation through the coupling of incident light with surface Plasmon modes (where the coupling occurs through the diffraction grating produced by the periodic patterning of the metal in the nanocavity architectures and by direct excitation of the local plasmon resonance of the nanoparticles) and interaction of the fluors with the metallic nanostructure (either by confinement within a metallic nanocavity or by proximity to a metallic nanoparticle) resulting in increased fluorescence yield. These enhancement mechanisms may produce more than an order of magnitude increase in fluorescence as compared to direct excitation on a transparent substrate.

Individual nanocavities have localized surface plasmon resonances that can undergo spectral shift upon change in the dielectric properties of the enclosed environment (i.e., target binding); when placed in specific spatial arrangements, collective oscillations can produce a narrower resonance. A measurable change in the position of the resonance peak can be obtained upon molecular binding within nanocavities. The spectral position of the resonance peak changes based upon the dielectric environment of the nanocavities. This label-free microarray detection target molecules may be achieved by using a conventional two-color scanner to measure the resonance shift in reflection upon molecular binding.

Nanocavity arrays significantly improve the signal to background ratio. Accordingly, nanocavity arrays can find great uses in many clinical, environmental, and industrial applications, thus offer fast, sensitive, real-time detection of target entities.

In typical real-time hybridization arrays (which are generally based upon evanescent wave excitation at a planar surface), excitation light covers the entire sensing zone area (as well as the entire array of zones), across which the probe molecules are spread uniformly. When bound target surface concentration is low (as it can be well before the end point of the hybridization kinetic reaction occurs or even at the end point for low concentration species), much of the excitation light is wasted, or, even worse, induces background signal from solvent or unbound species lying within the evanescent field. Without wishing to be bound by theory, real-time hybridization arrays, where the sensing zones are based upon sub-arrays of metallic nanostructures, solve this problem by two mechanisms: via selective surface chemistry, target molecules only bind to the nanostructures, about which excitation and emission enhancement of fluorescence occurs (by a total factor Mₜₒₜ); and the fill fraction η over which these enhancements occur is less than 100%, thereby providing isolation from sources of background fluorescence. These factors are nearly identical to the advantages that confocal and near-field techniques offer in single-molecule fluorescence microscopy (W. E. Moerner and D. P. Fromm, “Methods of single-molecule fluorescence spectroscopy and microscopy,” Review of Scientific Instruments 74, 3597-3619 (2003)), except that the nanoscopic detection sites are fixed (and arranged in sub-arrays within a detection zone) and the molecules of interest diffuse to the detection sites where they bind.

Arrays of metallic nanocavities may be used in clinical diagnostics, where a particular area of need is to improve the ability to rapidly determine the identity of pathogens responsible for a given infection. With respect to identification, there are clinical scenarios where similar signs and symptoms can result from infection by any of a multitude of pathogens, including viruses, bacteria and, in some instances, fungi. A common clinical example is pneumonia, where it would be desirable to test for up to ten different viruses and bacteria (e.g., Streptococcus pneumoniae, Influenza, etc.).

Another clinical example where multiple organisms can be causative is sepsis wherein a pathogen(s) is proliferating in the bloodstream. Currently, the mainstay technology of pathogen identification remains culture of the organism followed by identification by biochemical means (for bacteria) or staining with antibodies (for viruses or bacteria). Culture and identification of bacteria typically requires two or more days and virus culture can take one to several weeks. Significant advances with regard to reducing identification times have been achieved by nucleic acid amplification methods, notably the polymerase chain reaction (PCR). For many viruses and some bacteria, identification times have been reduced to less than one day. However, PCR methods in their current format remain limited in terms of their ability to simultaneously detect more than 6-8 pathogens. This limitation is due to the technical challenges associated with multiplexing PCR and a limited number of sufficiently spectrally distinct detection fluors that can be bound to oligonucleotide probes. In this context, there is substantial interest in and effort directed towards exploiting array based technologies wherein amplification technologies, such as PCR, are used to multiplex amplify various signature genetic regions from suspected pathogens followed by hybridization of the amplification products to an array containing complementary sequences. While technically attractive, a current bottleneck remains the length of hybridization time required to achieve sufficient signal above background, with most arrays requiring hybridization times on the order of hours to overnight (Y. Y. Belosludtsev, D. Boweman, R. Weil, N. Marthandan, R. Balog, K. Luebke, J. Lawson, S. A. Johnston, C. R. Lyons, K. O'Brien, H. R. Garner, and T. F. Powdrill, “Organism identification using a genome sequence independent universal microarray probe set,” Biotechniques 37, 654-660 (2004)).

Ideally, with infectious disease, physicians would like the result of pathogen identification within 2 hours of sample collection. There are three steps in molecular diagnostics: 1) sample preparation, 2) PCR, and 3) detection. Sample preparation takes roughly 15-30 minutes, while PCR can be performed in 30-60 minutes. In the case of infectious disease, this leaves roughly 30-75 minutes for detection, which is clearly not possible with current hybridization arrays. Nanocavity arrays as hybridization zones may provide fast and sensitive detection of multiple pathogens.

Another example of an application for nanocavity arrays is gene expression analysis (H. P. Saluz, J. Iqbal, G. V. Limmon, A. Ruryk, and W. Zhihao, “Fundamentals of DNA-chip/array technology for comparative gene-expression analysis,” Current Science 83, 829-833 (2002)). With their increased sensitivity (owing to real-time analysis and greatly improved signal to background ratio), nanostructure hybridization arrays can facilitate a new scientific avenue in expression analysis through the detection of less abundantly expressed genes, which currently cannot be studied by using endpoint or other real-time methods. This new capability could ultimately lead to better understanding of regulatory pathways, drug intervention, and the biological behavior of tumor cells, for example. Another avenue lies in the ability to analyze RNA populations directly (where even linear amplification steps can either be avoided entirely or minimized) where end-labeling of RNA would occur after a digestion step to cleave the native RNA to 20 to 100 bases; cleaving is performed to enhance the hybridization kinetics via increased diffusion.

Low-cost nanofabrication techniques can be applied such that the cost of producing the array itself may be no greater than the cost of producing a compact disc. For real time array analysis, low-cost and disposable microfluidic flow cells may be attached to the array in a hybridization unit, which can be compactly integrated with external optical excitation and parallel readout using imaging optics and a low-noise charge-coupled device camera or other suitable device. The coupling of excitation light is straightforward (comparable to the methods used in SPR biosensors), as a broad beam of light covers the entire array and the coupling is relatively insensitive to angle and wavelength (as opposed to standard SPR).

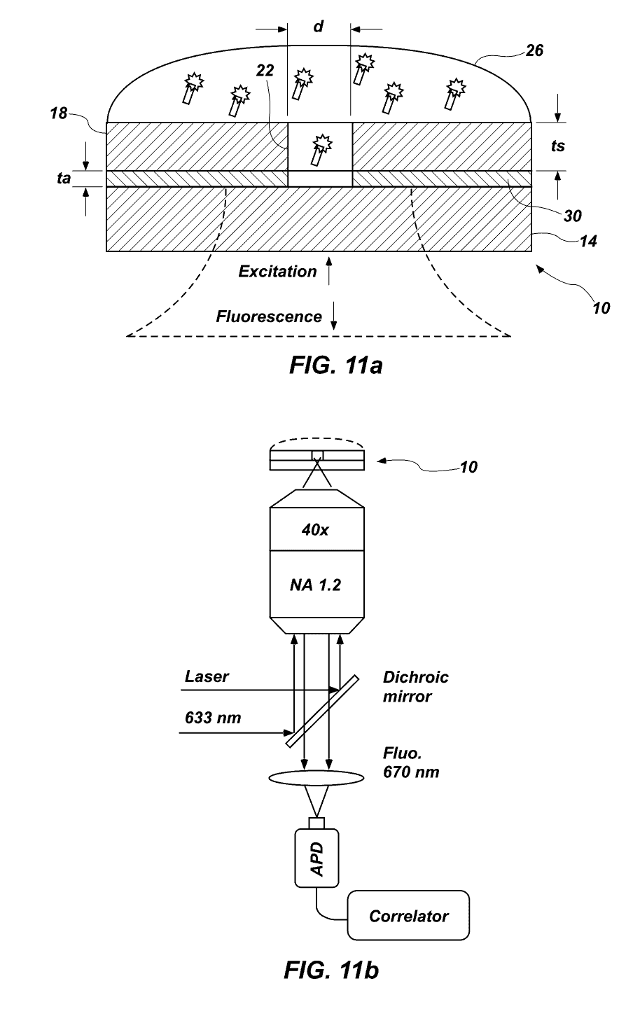

As a further description of the present technology, FIG. 11a shows a light enhancement device and/or detection enhancement device for a biological assay, indicated generally at 10, in an example implementation. The device includes a substrate 14 with a metallic layer (or second layer) 18 disposed over the substrate. The substrate 14 can be or can be formed of glass, quartz, another optically suitable (e.g., transparent) inorganic material, an optical plastic, a combination of any of the foregoing (as is the case in so-called “thin-film” waveguides, which include multiple layers), etc. The metallic layer 18 can be or can include gold, silver, aluminum, their alloys or combinations thereof. The metallic layer or film can be applied to at least one surface of the substrate by deposition techniques, lamination processes, etc. In one aspect, the metallic layer 18 can have a thickness ts between 50 to 200 nanometers. In another aspect, the thickness ts of the metallic layer can be 75 to 200 nanometers. In another aspect, the thickness ts of the metallic layer can be 75 to 125 nanometers. In another aspect, the thickness ts of the metallic layer can be less than 75 nanometers.

At least one nanocavity or an array of multiple nanocavities, represented by 22, extend into the metallic layer 18, and can extend all the way to the substrate 14. The nanocavities 22 each having a bottom and a sidewall laterally circumscribing or surrounding each nanocavity. Thus, the nanocavity is enclosed on the bottom and lateral sides, while being open at the top. The nanocavities can be formed in the metallic film by suitable processes (e.g., mask and lift-off processes (such as those used in semiconductor device fabrication), mask and etch processes (such as those used in semiconductor device fabrication), with a laser, etc.). The nanocavities may extend completely through the metallic film, with the underlying substrate being exposed therethrough. A lateral dimension (e.g., diameter) of each nanocavity may be about the same as the thickness of the metallic layer, although lateral nanocavity dimensions may differ from the thickness of the metallic layer. In one aspect, the nanocavities can have a lateral dimension d or diameter of 65 to 190 nanometers. In another aspect, the dimension or diameter d of the nanocavities can be about 100 to 140 nanometers. In another aspect, the dimension or diameter d of the nanocavities can be about 65 to 85 nanometers. In another aspect, the dimension or diameter d of the nanocavities can be about 120 to 160 nanometers. In another aspect, the dimension or diameter d of the nanocavities can be about 150 to 190 nanometers.

Nanocavities of virtually any shape may be formed. Examples of nanocavity shapes include, but are not limited to, round (e.g., circular, oval, elliptical, egg-shaped, etc.), quadrilateral (e.g., square, rectangular, parallelogram, trapezoidal, etc.), triangular, and other polygonal shapes. The nanocavities that are formed in a metallic film may all have substantially the same shapes and dimensions, or a variety of shapes and/or dimensions of nanocavities may be included in the metallic film of a biomolecular assay.

The nanocavities may be arranged in such a way that facilitates the coupling of incident light into surface modes, or waves, on the metallic film, which surface modes can constructively interfere within the nanocavities. For example, when incident light is to be directed from the substrate, or back side of the biomolecular assay, and fluorescence is to be detected at a location adjacent to the opposite, top surface of the biomolecular assay (i.e., the surface by which the metallic film is carried), the metallic film prevents excitation of fluorophores in the bulk solution 26, which is located over the metallic substrate. As another example, when incident light is directed toward the biomolecular assay from a location over the metallic film and detection occurs at a location adjacent to the back side of the substrate, although marker molecules that remain within solution may undergo a change in state (e.g., fluorescence by fluorescent marker molecules), the marker molecules that remain in solution over the metallic film remain substantially undetected. This is because light emitted from a location above the metallic film does not pass through the metallic film and since the size of each nanocavity may be too small for fluorescent light emitted from locations over the surface of the metallic film to pass therethrough. Fluorescent light generated within the nanocavities does exit the nanocavities, however, and is enhanced by the materials from which the nanocavities are formed, as well as by the configurations and dimensions of the nanocavities.

In specific applications, however, fluorescence signals originating from fluorescent species lying outside of the cavity may be a concern. For example, these signals may increase background or noise of an assay and thus compromise the sensitivity and/or precision of the assay. Partial or complete isolation of fluorescence signals originating from fluorescent species lying outside of the cavity can be obtained by either narrowing the fluorescence collection angle or by passivating the surfaces of the metallic film.

The shapes of the nanocavities may be configured to optimize signal amplification. It has been discovered that nanocavities of a variety of shapes, including circular, square, and triangular, provide a good degree of radiative, or signal, enhancement, depending upon the dimensions (e.g., diameters of circular nanocavities, edge lengths of square and triangular nanocavities, etc.) of the nanocavities. Square nanocavities may provide better signal enhancement than circular nanocavities, while triangular nanocavities may provide even greater signal enhancement.

Surfaces of the biomolecular substrate may be passivated to prevent capture molecules (e.g, bait molecules) from adhering, or being immobilized, to undesired locations thereof. The surfaces of the metallic film may be passivated, for example, with polyethylene glycol (PEG)-thiol, another metal (e.g., gold)-selective thiol molecule, or any other material that prevents capture molecules from being immobilized to the metallic film, or reduce immobilization of capture molecules to the metallic film. Thus, the capture molecules are instead immobiLized to the surface of the substrate exposed to and located within or adjacent to the nanocavities. Alternatively, the exposed surfaces of the substrate may be passivated (e.g., with PEG silane) to prevent capture molecules from adhering to the substrate and, rather, causing the capture molecules to be immobilized only to the metallic surfaces. As another alternative, a major surface of the metallic film may be covered with a coating film (e.g., another transparent film), and the exposed surfaces of the coating film, as well as surfaces of the substrate that are exposed through the coating film and the metallic film, may be passivated, causing capture molecules to adhere only to the unpassivated exposed edges of the metallic film, which form part of the surface of each nanocavity.

In addition, capture molecules can be disposed within the nanocavities. Capture molecules are introduced into the nanocavities and immobilized to surfaces of the nanocavities, the substrate, or both. The capture molecules are specific for one or more analytes of interest. Target or capture molecules (e.g., probe oligonucleotides) of an assay may reside on the sidewalls, the bottom surfaces, or both the sidewalls and the bottom surfaces of the nanocavities. One molecular species (i.e., the target oligonucleotides or analyte, which is fluorescently labeled) specifically bind to the capture oligonucleotides through hybridization. An optically-transduced, real-time signal in proportion to the number of bound target oligonucleotides are detected from the side of the sensor array opposite to the side on which the sample solution is introduced, thereby providing isolation from unbound species, which may represent a significant fraction of the detected signal in a washless assay. Fluorescence from unbound species do not penetrate the opaque metal except at the nanocavities. By measuring the hybridization kinetics in real-time, nonspecific binding may be factored out as well.

To prevent capture molecules from binding to undesired locations of the nanocavities, the surfaces of the metallic film or the surface of the substrate may be passivated. For example, a surface of the metallic film may be passivated with polyethylene glycol (PEG)-thiol. Thus, the probe oligonucleotides (i.e., capture molecules) are selectively attached to the bottoms of the nanocavities. On the other hand, the exposed surface of the substrate may be passivated (e.g., with PEG silane) to prevent capture molecules from adhering to the surface of the substrate and, rather, causing the capture molecules to be selectively attached to the sidewalls of the nanocavities. Alternatively, to facilitate coupling of the capture molecules to the sidewalls of the nanocavities, a thin (˜20 nm) cover layer of SiO₂ is deposited to cover the top metal surface. With this structure, the only exposed substrate surface is the inside walls of the nanocavities, to which selective derivatization of capture molecules is performed. A layer (e.g., 5 nm) of Al or Cr, may be used to promote adhesion of the SiO₂ layer. The passivation layer disposed over the metallic layer is capable of preventing adsorption of a molecule of interest onto the metallic layer.

Surfaces of nanocavity substrates may also be modified for covalent or noncovalent immobilization of capture molecules. For example, SiO₂ and Si₃N₄ surfaces of nanocavity substrates may be modified with a reactive species, e.g., epoxysilane. After surface modification with a reactive species, coating of the capture molecules may be performed. For example, amine-modified nucleic acid probes may be spotted on epoxysilane-modified surfaces, and reactions of the amine groups and the epoxy groups cause the amine-modified nucleic acid probes covalently linked to the surface of the substrate.

In addition, blocking layer of lossy material, such Cr, Ti and/or Al, can be disposed on or over the metal layer to resist light from transmitting through the device and/or metallic layer and into the solution.

Furthermore, an adhesion layer (or first layer) 30 adheres the metallic layer 18 to the substrate 14. The adhesion layer is disposed closer to the substrate than the metallic layer. The nanocavities can extend through the adhesion layer to the substrate. The adhesion layer has a first layer thickness to and a material, the metallic layer has a second layer thickness ts and a material, and the at least one cavity has a cavity diameter d and a cavity shape adapted to enhance transmission of light through the at least one nanocavity; and/or to enhance a signal representative of an amount of at least one analyte present in a sample. As discussed above, it has been found that the plasmonic enhancement of single-molecule fluorescence has a strong dependence on the nature, such as material and thickness, of the adhesion layer. In one aspect, the adhesion layer can be or can include titanium dioxide TiO₂. In another aspect, the adhesion layer can be or can include titanium dioxide and chromium oxide and combinations thereof. In one aspect, the thickness ta of the adhesion layer 30 and the diameter d of the cavity 22 can have a ratio in the range of 1:4 to 1:100. In another aspect, the thickness ta of the adhesion layer 30 and the diameter d of the cavity 22 can have a ratio in the range of 1:4 to 1:40. In another aspect, the thickness ta of the adhesion layer 30 and the diameter d of the cavity 22 can have a ratio in the range of 1:4 to 1:30. In another aspect, the thickness ta of the adhesion layer 30 and the diameter d of the cavity 22 can have a ratio in the range of 1:5 to 1:30. In another aspect, the metallic layer can be or can include aluminum, the thickness ta of the adhesion layer 30 and the diameter d of the cavity 22 can have a ratio in the range of 1:4 to 1:40. The thickness ta of the adhesion layer 30 can be between 2 to 15 nanometers. In one aspect, the adhesion layer causes an improvement in light transmission by a factor of at least 3.

In addition, the at least one cavity can comprise a tapered sidewall with an angle. The angle of the tapered sidewall with respect to a surface parallel to the substrate can be sufficiently different than 90° to provide an enhancement of the transmission of light through the at least one cavity, an enhancement of the intensity of light within the at least one cavity, or both, that is greater than the enhancement if the angle was 90°. Furthermore, at least one change in a sidewall within the at least one cavity can including a change in angle, a change in material, a change in width, or combinations thereof sufficient to provide an enhancement of the transmission of light through the at least one cavity, an enhancement of the intensity of light within the at least one nanocavity, or both, that is greater than the enhancement without the change in the sidewall.

The biomolecular assay may be used in an assay system or technique that employs fluorescence detection techniques. Such a system also includes a source of electromagnetic radiation and a detector. The source is configured to emit electromagnetic radiation of one or more wavelengths, or “incident light,” that excites fluorescent dye molecules that are to be used in the system, and oriented to direct the radiation onto the nanocavities or into the substrate, The incident light may be in the form of light transmitted from a source, an evanescent field generated as light is directed into and internally reflected within a substrate or transparent film that comprises a waveguide, or a combination thereof. Radiation can penetrate the nanocavities directly and, optionally, due to constructive interference that may occur because of the arrangement of the nanocavities, thereby exciting species within the nanocavities, or radiation may be internally reflected within the substrate, generating an evanescent field at one or more surfaces thereof. The incident light excites fluorescent dye molecules that are immobilized (directly or indirectly, depending upon the assay binding technique (e.g., a sandwich-type assay, a binding competition assay, etc.) employed relative to capture molecules within the nanocavities. Fluorescent dye molecules within the nanocavities are excited and, thus, emit electromagnetic radiation. The electromagnetic radiation is enhanced by the nanocavities and the metallic substrate. It is then detected by the detector. An aperture associated with the detector may tailor the angle of a collection cone of radiation emitted by the fluorescent dye molecules.

The biomolecular assay may be used with known mass detection processes. As an example, a reference analyte of known concentration and analyte within a sample, which has an unknown concentration, may be labeled with different marker molecules (e.g., fluorescent molecules that emit different wavelengths, or colors, of light) and their binding to capture molecules that have been immobilized within the nanocavities compared to provide an indication of the amount of analyte in the sample. The affinities of the reference analyte and the sample analyte for the capture molecule, which may be known, may be the same or different.

The invention is further described in the following non-limiting examples, which are offered by way of illustration and are not intended to limit the invention in any manner.

### EXAMPLES

### Example 1

### Biosensing Based Upon Molecular Confinement in Metallic Nanocavity Arrays

Liu et al. (Y. Liu, J. Bishop, L. Williams, S. Blair, and J. N. Herron, “Biosensing based upon molecular confinement in metallic nanocavity arrays,” Nanotechnology, vol. 15, pp. 1368-1374, 2004) describes the basis for an affinity biosensor platform in which enhanced fluorescence transduction occurs through the optical excitation of molecules located within metallic nanocavities. The contents of Liu et al. are hereby incorporated herein, in their entireties, by this reference. The nanocavities of Liu et al. are about 200 nm in diameter, are arranged in periodic or random two-dimensional arrays, and are fabricated in 70 nm thick gold films by e-beam lithography using negative e-beam resist. It has been shown that both periodic and randomly placed metallic nanocavities can be used to enhance the fluorescence output of molecules within the cavities by about a factor of ten. In addition, the platform provides isolation from fluorescence produced by unbound species, making it suitable for real-time detection, for example, real-time detection of 20-base oligonucleotides in solution.

### Example 2

### Enhanced Fluorescence Transduction Properties of Metallic Nanocavity Arrays

Liu et al. (Y. Liu, F. Mandavi, and S. Blair “Enhanced fluorescence transduction properties of metallic nanocavity arrays,” IEEE Journal of Selected Topics in Quantum Electronics 11, 778-784 (2005)) describes fluorescence enhancement of molecular species bound within metallic nanocavities. The contents of Liu et al. are hereby incorporated herein, in their entireties, by this reference. The nanocavity structures of Liu et al. possess a number of desirable properties for real-time microarrays, such as localization of excitation light within the nanocavities, strong isolation from fluorescence produced by unbound species, and an apparent increase in fluorescence yield for bound species. Experimental measurements show nearly a factor of two increase in excitation intensity within the nanocavities, and factor of six increase in yield. An electromagnetic model of a dipole within a nanocavity shows an increase in radiative output consistent with estimated yield, and also verifies the strong fluorescence isolation from species lying outside the nanocavity.

### Example 3

### Biosensing Based Upon Molecular Confinement in an Array of Metallic Nanocavities

The following study is designed to optimize and validate a hybridization array platform based upon sub-arrays of metallic nanocavities. These nanocavity arrays are conducive to real-time measurement of hybridization kinetics, are scalable to large hybridization array formats, and possess significantly improved molecular sensitivities to enable rapid screenings in a clinical setting.

To determine molecular sensitivity in a real-time assay, derivatization of the nanocavities either occurs at the bottom (quartz) surface or at the gold sidewall to avoid the binding of species in solution to the top surface. Therefore, two nanocavity array embodiments are studied for selective derivatization of either the exposed quartz surface or the nanocavity sidewall. The first embodiment (Type I) is shown schematically in FIG. 3 (c). These samples are passivated with mPEG-thiol. The second embodiment (Type II) is shown in FIG. 3 (d) and consists of a thin (^{˜}20 nm) overcoat of SiO₂. A ^{˜}5 nm layer of Al or Cr is used to promote adhesion of the SiO₂. With this structure, the only exposed gold surface is the inside walls of the nanocavities, to which selective derivatization is performed.

Previous studies identified two fluorescence enhancement effects associated with nanocavity arrays—yield enhancement and surface plasmon excitation enhancement. These enhancements are studies in more detail using the two embodiments that allow for independent and direct measurement of enhancement factors—an embodiment with molecules located at the bottom (Type I) and an embodiment with molecules located on the sidewall (Type II). The enhancement factors—with additional denotation M^{bottom} and M^{side} for the Type I and II embodiments—are calculated based upon the respective surface areas—A^{bottom}=π2/4 and A^{side}=2πdh—where d and h are the nanocavity diameter and height, respectively. When h>d/2, the surface area of the sidewall is greater than the surface area of the bottom. The enhancement factors associated with cavity-enhanced fluorescence in random arrays, which result in increased fluorescence yield given by M^{bottom}_{cav} and M^{side}_{cav}, are determined. The surface plasmon excitation enhancement of fluorescence in periodic arrays given by M^{bottom}_{SP} and M^{side}_{SP} are also determined. Individual detection zones, which are 75 μm×75 μm in size, are fabricated to characterize nanocavity enhancement and optimize surface plasmon excitation enhancement. These zones are arranged in a 3×3 array for the studies of molecular sensitivity in a real-time assay. E-beam lithography other suitable techniques may be used to fabricate individual detection zones. Another fabrication technique that may be used is nanoimprint lithography (S. Y. Chou, P. R. Krauss, and P. J. Renstrom, “Nanoimprint lithography,” Journal of Vacuum Science & Technology B 14, 4129-4133 (2004)), in which arbitrary patterns can be written using e-beam or focused ion beam lithography on a master, then the master used to stamp out multiple copies (in an analogy to the process used for CD's and DVD's, which are basically a thin metal layer surrounded by dielectric layers).

### Example 3.1

### Characterization of Nanocavity Enhancement

In order to characterize the enhancement in fluorescence yield produced by molecular confinement in a metallic nanocavity, and verify that the majority of the cavity enhancement results from an increase in fluorescence collection efficiency, detailed characterizations of derivatized nanocavities are undertaken.

Development and characterization of nanocavity derivatization procedures. Two different passivation procedures are employed for selective derivatization of the nanocavities, for which labeled avidin monolayers are formed. The passivation of the gold surfaces using mPEG-thiol follows the reference (K. L. Prime and G. M. Whitesides, “Adsorption of proteins onto surfaces containing end-attached oligo(ethylene oxide): a model system using self-assembled monolayers,” Journal of the American Chemical Society 115, 10714-10721 (1993)) for the Type I embodiment, such that monolayer formation occurs only on the bottom of the nanocavities. A similar procedure is developed for the Type II embodiment (i.e., with the SiO₂ overcoat) to passivate the dielectric surfaces, thus allowing monolayer formation only on the exposed gold sidewalls. For Type II, dielectric surface passivation is performed using mPEG-silane. Introduction of the labeled avidin solution then allows monolayer formation on the exposed gold surfaces. An alternate method of selectively derivatizing the side-walls may also be employed, which follows a procedure developed with biotinylated thiol (D. M. Disley, D. C. Cullen, H. X. You, and C. R. Lowe, “Covalent coupling of immunoglobulin G to self assembled monolayers as a method for immobilizing the interfacial-recognition layer of a surface Plasmon resonance immunosensor,” Biosensors and Bioelectronics 13, 1213-1225 (1998)), which binds strongly to gold surfaces. Cy-5 labeled neutravidin is then be used for the monolayer, where the neutravidin does not undergo a charge-charge interaction with the dielectric surfaces, but interacts strongly with the biotin layer via two of its biotin binding sites. This procedure is tested with two reference surfaces, a quartz surface which, after derivatization, should exhibit no measurable fluorescence, and a gold surface which should exhibit strong fluorescence. During the initial avidin labeling step, there is less than 1 Cy-5 molecule per labeled avidin, on average. This avoids any effects of energy transfer among a group of Cy-5 molecules clustered on a single avidin molecule, which would complicate the interpretation of cavity enhancement of fluorescence yield. The labeled avidin solution is then diluted using pure 1 μM avidin to the final concentration.

Previous studies suggest that the gold passivation procedure performed on a quartz reference surface results in a reduction in avidin surface concentration as compared to a quartz control surface without the passivation step. The reference samples are further characterized in terms of total fluorescence emission as well as ellipsometry studies to compare extinction at 280 nm (650 nm), which is directly proportional to the avidin (Cy-5) surface concentration. When the surface concentrations differ between reference and control, it is likely that the reference surface contains residual mPEG-thiol (on a quartz reference surface) or mPEG-silane (on a gold reference surface), which can be detected either with ellipsometry or XPS measurements. It may be that reduced surface concentration on the desired surface is an unavoidable consequence of the passivation process.

Because these passivation procedures require multiple steps involving mass transport into and out of nanoscale volumes, it is important that the final derivatized surfaces be fully characterized. One motivating factor behind the characterization studies is the determination of bound avidin surface concentration within the nanocavities, which cannot be performed using the standard XPS or ellipsometry techniques. Characterization is performed with two methods, initially using low aspect ratio h/d^{˜}0.1 such that capillary effects play a negligible role in mass transport. After each successful characterization step, the aspect ratio is increased, with the goal of successfully derivatizing nanocavities with h/d>1. The first characterization method employs radiolabeled avidin (with ¹²⁵I) for the monolayer. The labeling procedure uses Enzymobeads (Bio-Rad Labs). Before labeling, sodium azide is removed from the avidin solution using Sephadex G-25 (Pharmacia) as sodium azide is a potent inhibitor of lactoperoxidase. The following reagents is added to an Enzymobead reaction vial: (1) 50 μL of 0.2 M phosphate buffer (pH 7.2); (2) 500 μL avidin solution; (3) 1 mCi Na¹²⁵I; (4) 25 μL of 2% glucose. This mixture is allowed to react for 40 min at room temperature. Unreacted Na¹²⁵I is removed by gel filtration (Sephadex G-25). Radiolabeling efficiency (RE) is determined by precipitating the labeled protein with 20% trichloroacetic acid (TCA) in presence of BSA as a carrier, and calculated using the following equation

RE=(CPMₛₒₗᵤₜᵢₒₙ−CPMₛᵤₚₑᵣ)/CPₛₒₗᵤₜᵢₒₙ (1)

where CPMₛₒₗᵤₜᵢₒₙ is the number of counts in 5 μL of the labeled protein solution before TCA precipitation and CPMₛᵤₚₑᵣ is the number of counts in 5 μL supernatant. Samples are counted using a Beckman Model 170M liquid scintillation counter. The specific activity of the iodinated protein is then calculated with the additional information of protein concentration determined from a UV-visible spectrophotometer at 278 nm. The radiolabeled avidin monolayer is formed using the same procedures as described previously. Radioassay is then used to determine the surface concentration of the immobilized avidin. Samples are counted for 1 min and the surface concentration calculated from

Γ=(CPM/SA)/A_{surf} (2)

where CPM is counts per minute, SA is the specific activity, and A_{surf} is the effective surface area, which is just the total area of the nanocavities. The surface concentrations from nanocavity array samples are compared to reference samples that have undergone the same surface modification steps. The second characterization method involves using avidin labeled with metallic nanoparticles which are roughly 2 nm in size. Conjugation with metallic nanoparticles provides contrast under scanning electron microscope imaging, such that the location of the avidin monolayer and uniformity within the nanocavities can be directly determined and compared to the reference sample. Gold nanoparticle-avidin conjugates are available commercially. A high-resolution (^{˜}2 nm) field-emission SEM is employed for these measurements.

In situations where the surface concentration within the nanocavities is not as high as that for the reference samples (which may occur as h/d→1), the parameters of the derivatization procedures are adjusted. For example, one problem that may occur is that the wash step after passivation may not completely clear the volume of the nanocavities, which could cause interference with avidin monolayer formation. Possible ways to solve this would be to lengthen the duration of the wash step and/or perform the wash step at elevated temperature. Another issue that may arise is that of air bubbles becoming trapped within some of the nanocavities, preventing surface modification. If this occurs, the samples in buffer solution in a side-arm flask are degassed to promote wetting of the nanocavities before derivatization.

Study of reproducibility. A series of measurements are made to determine the reproducibility of the enhancement factor across multiple samples. Measurements are performed on 20 different samples of each embodiment (Type I and Type II), five periodic array samples and five random array samples fabricated using the etching procedure, and five of each pattern fabricated using the lift-off procedure. The nanocavity size is 150 nm diameter with 1 μm spacing, and the fluorescence outputs are measured relative to coated quartz substrates. The standard deviations from these measurements σref reflect variations in pattern fabrication, monolayer coating, and optical alignment and detection. When combined with the standard deviations of subsequent measurements described below (σmeas), it can be determined whether or not a change in a measured quantity such as the enhancement factor can be considered statistically significant. Using the nanofabrication process based upon metal etching, we obtained σref≦7% and 11% for d=200 nm for periodic and random arrays, respectively. The deviation is greater for d=150 nm, but with the new lift-off process, it is expected to reduce σref over the etching process for the same d. For subsequent studies, a nanofabrication process is chosen based upon the one that produces the smallest variation.

Fluorescence yield enhancement by a nanocavity. The following procedures are designed for determining the origin of the fluorescence yield enhancement by molecular confinement within a metallic nanocavity by using random arrangements of nanocavities where surface plasmon excitation enhancement is suppressed. The net increase in fluorescence yield is given by the factor M_{cav}=M_{rad}·M_{coll}, where M_{cav} is the fluorescence enhancement factor from the random arrays, M_{rad} is increase due to an increase in the radiative rate, and M_{coll} results from an increase in fluorescence collection efficiency. Previous studies using random nanocavity arrangements indicated that M_{cav} ranged from about 10 for 120 nm diameter cavities to about 7 for 200 nm diameter cavities, with a predicted peak for 150 nm diameter. This observation suggests a strong influence of the nanocavity geometry on the emission properties of the fluors, most likely based upon the nanocavity aspect ratio h/d, where h is the height. This effect is studied experimentally by using nanocavity diameters ranging from 50-250 nm in diameter and comparing with results from simulation.

The total fluorescence enhancement factor due to the nanocavity is calculated for both Type I and Type II embodiments by measuring fluorescence output from random nanocavity arrangements relative to reference samples to give M_{cav}. These measurement rely on direct excitation of fluors within the nanocavities by incident light. Since the collection efficiency factor M_{coll} is difficult to measure directly, it is instead indirectly determined from the measurements of M_{cav} and M_{rad}. In order to estimate the fluorescence yield enhancement due to modification of the radiative rate, direct fluorescence lifetime measurements is performed with a time-correlated single photon counting system, where instead of imaging a single nanocavity at a time, a roughly 16 μm spot size is excited and imaged. This makes the experiments easier to perform and is one reason for using the random nanocavity arrays. With this method the lifetime can be measured for molecules on the bottom (Type I embodiment, giving M^{bottom}_{rad}) and on the side-walls (Type II embodiment, giving M^{side}_{rad}). For fluors on the sidewall, the fluor to metal separation is the thickness of one avidin molecule (approximately 5 nm), while for fluors on the bottom, the average separation is d/6>15 nm. Experiments with nanoparticles (A. Wokaun, H.-P. Lutz, A. P. King, U. P. Wild, and R. R. Ernst, “Energy transfer in surface enhanced luminescence,” Journal of Chemical Physics 79, 509-514 (1983); J. Malicka, I. Gryczynski, Z. Gryczynski, and J. R. Lakowicz “Effects of fluorophore-to-silver distance on the emission of cyanine-dye-labeled oligonucleotides,” Analytical Biochemistry 315, 57-66 (2003)) suggest maximum fluorescence enhancement due to increase in radiative rate for fluor to metal separation of about 9 nm. It is anticipated that the net cavity enhancement is maximized for aspect ratio h/d 100/150=0.67, due mainly to increase in collection efficiency. These measurements can identify the largest enhancement factor that can be obtained given practical limitations in nanofabrication and derivatization. For each embodiment, rigorous electromagnetic simulation (using FEMLAB, a commercial finite element method differential equation solver) of the radiative properties of a molecular dipole (using all polarization orientations) is performed at various positions within a metallic nanocavity in order to verify the experimentally derived M_{coll}. For example, for the Type II embodiment, dipoles are placed along the sidewall of the nanocavity, spaced 5 nm from the metal surface. The total radiative output from the nanocavity is compared to the output produced by the same dipole on a quartz surface to estimate M_{coll}. An average radiative enhancement is then be calculated based upon the M_{coll} values produced for the three orthogonal dipole orientations, and dipole positions distributed along the bottom of the nanocavity (Type I) or along the sidewall (Type II).

The importance of these measurements is threefold: 1) to identify the spatial region of the nanocavity (i.e., Type I or Type II embodiment) where the greatest increase in yield due to cavity enhancement occurs, 2) to determine the contributions to net yield by radiative rate enhancement and collection efficiency enhancement, and 3) to determine the maximum net yield given the practical limitations in nanocavity aspect ratio, where h/d=0.67 is likely optimal with >10. The net cavity enhancement for a given fluor is independent of excitation intensity, and determines the photobleaching limited transduction sensitivity enhancement over detection performed on a quartz substrate, as done for end-point detection methods. This part of the study determines the nanocavity geometry (in terms of the values of diameter d and height h) that is applied for subsequent studies.

### Example 3.2

### Optimization of Surface Plasmon Excitation Enhancement

The following procedures are designed to optimize the fluorescence excitation enhancement by surface plasmon coupling M_{SP} in periodic arrays using Type I and Type II embodiments. Previous studies suggest that the surface-plasmon enhancement factor is nearly uniform for the two embodiments, i.e., M^{bottom}_{SP}^{˜}M^{side}_{SP}.

The total fluorescence enhancement factor Mₜₒₜ is calculated by the ratio of normalized fluorescence output from the periodic array to the reference surface, while the surface plasmon excitation enhancement factor M_{SP} is given by the ratio of fluorescence between the periodic and random array of the same nanocavity diameter d and average spacing Λ. From these quantities, an apparent increase in fluorescence yield can be determined through the ratio M_{cav}=Mₜₒₜ/M_{SP}, as described herein earlier.

Previous studies indicate that the intensity enhancement factor due to surface plasmon coupling MSP is about a factor of two, but it is expected that with optimization of the geometrical parameters, this factor may increase to 7 or more (H. J. Lezec and T. Thio, “Diffracted evanescent wave model for enhanced and suppressed optical transmission through subwavelength hole arrays,” Optics Express 12, 3629-3651 (2004)). The origin of the excitation enhancement within the nanocavities results from the fact that under the condition of enhanced transmission (Y. Liu and S. Blair, “Fluorescence enhancement from an array of sub-wavelength metal apertures,” Optics Letters 28, 507-509 (2003)) energy is concentrated within the nanocavities. The periodicity of the nanocavities not only supports coupling incident light from free space into surface plasmon modes, but also modifies the propagation properties of the surface plasmon (I. I. Smolyaninov, W. Atia, and C. C. Davis, “Near-field optical microscopy of two-dimensional photonic and plasmonics crystals,” Physical Review B 59, 2454-2460 (1999)) through coherent scattering off the walls of the nanocavities, which results in constructive interference within the nanocavities. In these studies, the immobilized fluors act as local probes to the optical intensity buildup within the nanocavities. Surface roughness of the metallic nanocavity side-walls may manifest in the measurements of surface-plasmon excitation enhancement as the nanoscale roughness can serve as concentration points for light intensity buildup via the “lightning-rod” effect (A. V. Ermushev, B. V. Mchedlishvili, V. A. Oleinikov, and A. V. Petukhov, “Surface enhancement of local optical fields and the lightning-rod effect,” Quantum Electronics 23, 435-440 (1993)). Any manifestation should average out across a nanocavity as the scale of the surface roughness is less than the size of the nanocavity. This effect can be quantified indirectly by estimating the surface roughness within the nanocavities for the Type I embodiment (the Type II embodiment should be similar) and incorporating these estimates into numerical models of light propagation through nanocavity arrays. Numerical modeling with FEMLAB can be used to aid in optimizing the nanocavity array geometrical factors for excitation enhancement and for characterizing the effects of side-wall roughness on the measured enhancement factors. Surface roughness is estimated experimentally by cross-section analysis of nanocavities that are strongly elliptical, such that the minor axis is in the 50-250 nm range, but the major axis is many μm's in length to make dicing and polishing more reliable. A high-resolution SEM can be used for cross-section inspection with a resolution of about 2 nm.

Excitation enhancement by surface plasmon coupling. The following procedures are designed to measure the fluorescence enhancement by fluors on the nanocavity sidewall M^{side}_{SP} and by fluors at the bottom of the nanocavity M^{bottom}_{SP} to determine the relative excitation efficiencies. For each embodiment, the fluorescence enhancement relative to the reference sample is determined for random and periodic arrays for different values of average nanocavity spacing Λ, ranging from about 600 nm to about 1 These measurements are performed as a function of incidence angle, where the peak fluorescence corresponds to angles of peak transmission of the incident light owing to surface-plasmon coupling.

Incidence angle is changed only along the x-axis, so that the intersections of the coupling curves with the horizontal axis correspond to the angles of peak transmission (and peak fluorescence). These measurements result in a two-dimensional data set of M_{SP} for each embodiment versus A and incidence angle θ, from which values of peak fluorescence can be extracted. It can be predicted where maximal values may occur. In general, fluorescence enhancement should increase with decreasing A. In addition, there are discrete values of Λ for which increased fluorescence enhancement should occur due to the overlapping of two or more coupling orders. For example, comparing the situations where Λ=1 μm and 678 nm, it is expected to obtain greater enhancement for Λ=678 nm at 27° than for Λ=1 μm at 25°. The reason for this is that at the smaller spacing, coupling at 27° corresponds (by design) to two overlapping diffraction orders, so that two surface plasmon waves are excited, thereby increasing the intensity within each nanocavity on average.

Optimization of surface plasmon enhancement using cross-coupling. The surface plasmon modes at each of the metal interfaces in general have different propagation constants, such that usually only one of these modes (the one at the metal-air interface) plays a role in enhanced transmission. However, the modes at the two interfaces can be coupled together (or cross-coupled (R. W. Gruhlke, W. R. Holland, and D. G. Hall, “Surface-plasmon cross coupling in molecular fluorescence near a corrugated thin metal film,” Physical Review Letters 56, 2838-2841 (1986); R. W. Gruhlke, W. R. Holland, and D. G. Hall, “Optical emission from coupled surface plasmons,” Optics Letters 12, 364-366 (1987))) when the momentum of the periodic lattice matches the differences in momenta of the two modes:

K=2π/Λ=|k_{sp,1}−k_{sp,2}| (3)

Under this condition, both interfaces play a role in enhanced transmission, and it is predicted that fluorescence excitation enhancement can be improved. For a given excitation wavelength (such as 633 nm), there is only one value of Λ for which cross-coupling occurs, which is approximately 1150 nm for the first embodiment. However, as the difference in the refractive indices of the dielectrics at the two interfaces increases, the necessary value of A decreases. For Λ=678 nm, the necessary refractive index on the substrate side is 1.513. Surface plasmon cross-coupling can be achieved by controlling the refractive index at the substrate by depositing a thin film of silicon oxynitride (SiON) between the gold and quartz substrate. With SiON deposited by plasma-enhanced chemical vapor deposition, one can have continuous control over refractive index in the range 1.46 to 2.05 by adjusting the relative ratio of oxygen and nitrogen atoms. Similar designs are tested for the second embodiment, where the effects of the finite thickness of SiO₂ on the top surface must be taken into account in the propagation constant of the surface plasmon wave at that interface.

The importance of these optimizations of excitation enhancement (in conjunction with the characterization of nanocavity enhancement) is twofold: 1) to determine the embodiment (Type I or Type II) that has the greatest overall fluorescence enhancement (i.e., Mₜₒₜ=M_{SP}·M_{cav}, where 60 or more is expected), and 2) to determine the geometrical and refractive index properties that maximize M_{SP}. The optimal embodiment and geometrical parameters are carried-forward to the studies of molecular sensitivity in real-time assays.

So far, these studies are directly comparable to the sensitivities obtained by end-point readout by scanning or imaging, where for the same incident power upon each hybridization zone, a hybridization zone comprising a nanocavity array offers enhanced fluorescence by a factor of Mₜₒₜ>12. This means that ^{˜}Mₜₒₜ fewer bound molecules per zone can be detected, implying that hybridization can be performed in roughly Mₜₒₜ the time. However, this is only part of the story as significant benefits can be gained by going towards a real-time detection approach that enables direct measurement of hybridization kinetics. These benefits include quantitative determination of target concentration in solution (which is very difficult with end-point analysis), discrimination against non-specific binding and heteroduplex formation, and short time to result. In order to perform real-time detection, there must be strong isolation from fluorescence produced by unbound species. As will be shown as follows, metallic nanocavity arrays provide greater isolation (by more than a factor of 10) than other surface-selective techniques, which, combined with the fluorescence enhancements already described, make this technique highly suited for DNA-based clinical diagnostics.

### Example 3.3

### Determination of Molecular Sensitivity in Real-Time Assay

To determine molecular sensitivity in a real-time assay, real-time nucleic acid hybridization measurements are performed with a 3×3 array of hybridization zones, to verify that the metallic nanocavity arrays provide strong fluorescence isolation from unbound species in proportion to 1/η(which is an important consideration in any washless assay) and to determine assay time as a function of target concentration, even in situation where non-specific binding may be an issue. Validation studies relevant to clinical diagnostics are performed using multiple target species and controls. A second experimental setup is built using a low-noise cooled CCD camera in order to simultaneously image the 3×3 array. The nanocavity array embodiment (i.e., Type I or Type II) and geometrical parameters that produce the greatest fluorescence enhancements are used, as determined from studies described hereinbefore. The hybridization zones are spaced far enough apart from each other (about 1 mm) that manual spotting can be performed using a flexible Micromachined gasket with open wells to isolate one zone from another. The T3 polymerase promotor site is used as a model system; T3 5′-(AATTAACCCTCACTAAAGGG)-3′ and complementary anti-T3 are commercially available, and can be fluorescently labeled with Cy-5. A synthetic 60-mers is also employed in the validation studies. Sample solution containing fluorescently labeled target and non-target species is introduced to the surface using a flow cell (Y. Liu, J. Bishop, L. Williams, S. Blair, and J. N. Herron, “Biosensing based upon molecular confinement in metallic nanocavity arrays,” Nanotechnology 15, 1368-1374 (2004)) (see FIG. 4), which resides on the top surface of the nanocavity samples.

Nanocavity derivatization with anti-T3 probe. The following procedures are designed to characterize the immobilization of capture oligonucleotides (anti-T3 for these studies) within the nanocavities. Formation of the avidin monolayer is followed by a solution of 0.15 μM 5′-biotinylated anti-T3 which self-assembles on top of the avidin-coated surface (J. N. Herron, S. zumBrunnen, J.-X. Wang, X.-L. Gao, H.-K. Wang, A. H. Terry, and D. A. Christensen, “Planar waveguide biosensors for nucleic acid hybridization reactions,” Proceedings SPIE 3913, 177-184 (2000)). Derivatization procedures are characterized using radio-labeled anti-T3 to determine probe surface concentration and optimize the procedure. For reproducibility purposes, these procedures are implemented identically in all 9 zones of the 3×3 array.

Radiolabeled oligonucleotides are prepared by end labeling with (³²P)phosphate. For determining the surface concentration of immobilized capture oligo (i.e., anti-T3), 5′-biotinylated oligos are labeled with (α-³²P)ATP using terminal transferase. This enzyme adds (³²P)AMP to the 3′ end of the oligo. A commercial 3′ end labeling kit (Perkin Elmer) is used to perform the reaction. The extra adenosine group is not expected to interfere with binding of the labeled oligo to the immobilized avidin monolayer. Radiolabeling efficiency is determined using a similar procedure as described before with equation (1). The oligo concentration is determined using a UV-vis spectrophotometer at 260 nm. After self-assembly of the radiolabeled anti-T3 onto the avidin monolayer, the probe surface concentration is determined according to equation (2) using the radioisotope detector. The surface concentration within the nanocavity array is compared to that obtained for a planar reference sample. The derivatization procedure for the nanocavities may need to be adjusted as a result of this comparison; for example, if the nanocavity surface concentration is lower, then it is necessary to increase the concentration of anti-T3 in solution from 0.15 μM before the self-assembly step. Before the real-time hybridization experiments are performed, a calibration between bound surface concentration and fluorescence intensity is performed. Using radiolabeling one can know the bound probe concentration. One can then perform the same surface modification procedures with Cy-5 3′ end-labeled anti-T3 (with labeling ratio determined by UV-vis absorption) to allow direct relation between measured fluorescence intensity and bound concentration. This relationship can be used in conjunction with the two-compartment model to determine the detection limits in terms of the number of bound target molecules and to optimize the probe concentration.

Determination of signal to background ratio. The following procedures are designed to verify fluorescence isolation from unbound species and to determine the detection sensitivity taking into account non-specific binding. Because detectable fluorescence can only be produced from within a nanocavity, random variation in fluorescence from non-target molecules only occurs when those molecules randomly diffuse into and out of the nanocavity (although some fraction may produce a signal due to non-specific binding). The nanocavity surface area represents a fraction η of the total zone area (η^{˜}2-10%), so that the background signal from unbound species should be less by a factor of approximately 1/η(^{˜}10-50) than in other washless, surface selective fluorescence sensors such as a planar waveguide or fluorescence-SPR where the sensing surface represents 100% of the zone area. Again, these studies are performed with the 3×3 array where all zones are derivatized with anti-T3 probe.

Because the transduction area in the nanocavity architectures is so small, diffusion of the target molecules into the sensing regions is slightly slower than if the transduction area were 100% of the sensing area. The first step is then to study hybridization kinetics of the labeled target as a function of target concentration in solution, as compared to a planar waveguide. Target oligos (T3), labeled at the 5′ end with Cy-5 dye are prepared in solution with a concentration Cₙ, where n is the trial number. Typical Molar concentrations range from 10⁻⁸ to 10⁻¹². When introduced into the flow cell, T3 hybridizes to probe oligos on the capture monolayer and form duplex DNA. The hybridization kinetic curve 74 is measured for each Cₙ through the time dependence of the fluorescence excited by light intensity within each nanocavity (as shown in FIG. 5). By comparing the kinetic curves between the two nanocavity architectures and the waveguide, with greatly different fill fractions, the increase in diffusion time can be estimated via the parameter k_{M}, as defined subsequently. In addition, optimization of immobilized probe concentration is performed by maximizing binding rate. One can analyze the kinetic curve using the two-compartment model (D. G. Myszka, X. He, M. Dembo, T. A. Morton, and B. Goldstein, “Extending the range of rate constants available from BIACORE: interpreting mass transport-influenced binding data,” Biophysical Journal 75,583-594 (1998)):

dC(t)/dt=1/hᵢ{−k_{α}C(t)(R_{T}−B(t)+k_{d}B(t)+k_{M}(C_{T}−C_{T}−C(t))} (4)

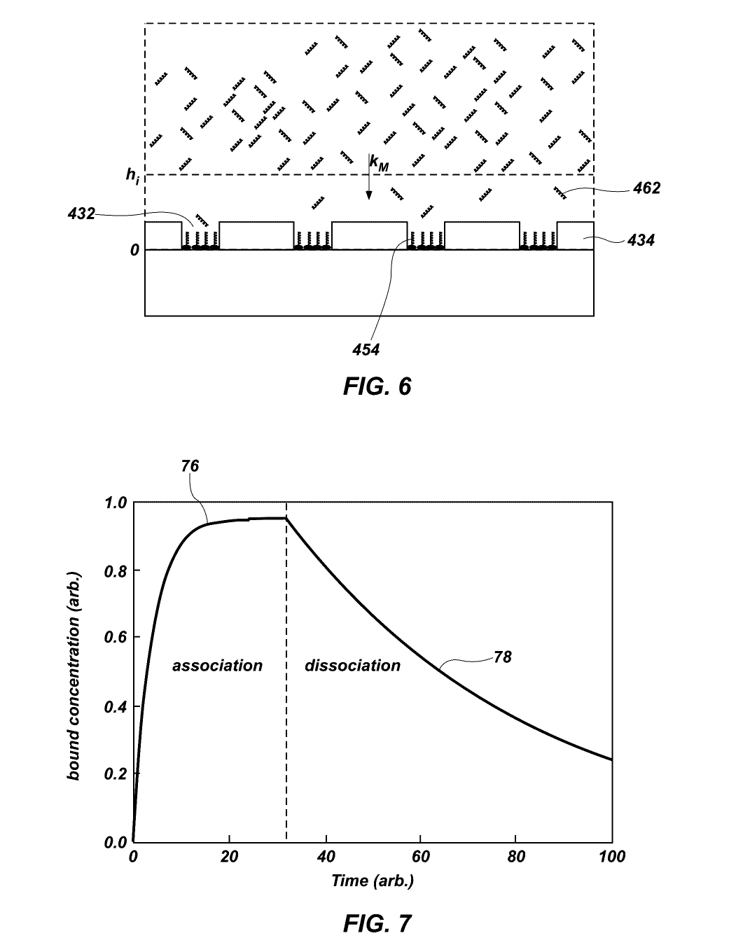

dB(t)/dt=k_{α}C(t)(R_{T}−B(t))−k_{d}B(t) (5)

where hᵢ is the height of the lower compartment where significant target depletion can occur, k_{α} is the association rate, k_{d} is the dissociation rate (which typically can be ignored for specific binding at room temperature), k_{M} accounts for mass transport between the upper and lower compartments, C(t) is the target concentration in the lower compartment, R_{T} is the probe concentration, and C_{T} is the target concentration in the upper compartment (which is assumed constant due to injection from the flow cell). These parameters are illustrated in FIG. 6 showing nanocavities 432 having capture molecules 454 for identifying species 462. The purpose for using the two-compartment model is that, through the fitting constant k_{M}, the effect of mass transport to the sparse array of detection sites can be determined and compared to a planar waveguide sensing modality in which the fill fraction is 100%. In order to differentiate the effects of non-specific binding, the two-compartment model is modified to describe the binding of two species to the surface with association constants k_{α1} and k_{α2}. In this case, two bound concentrations B₁(t) and B₂(t) are obtained, and the density of available binding sites is given by R_{T}−B₁(t)−B₂(t). One may also have to incorporate the effects of dissociation for the non-specific species. The ultimate goal for clinical diagnostics is to use the two-compartment model to analyze the kinetic curve at each hybridization zone to obtain the unknown concentration C_{T} of the desired target in solution.

The next step is to perform kinetic measurements using a second labeled sequence of the same length as T3/anti-T3 to determine the kinetic coefficients in the two-compartment model for non-specific binding (NSB). The sequences of these “background” oligos are chosen so as not to specifically bind to either the target or probe molecules. These background oligos diffuse into the nanocavities and produce a random background signal, which could mask the kinetic curve produced by bound species, and may also non-specifically bind, which produces a signal that mimicks the kinetics of the target species (but with a different rate and equilibrium value (H. Dai, M. Meyer, S. Stepaniants, M. Ziman, and R. Stooughton, “Use of hybridization kinetics for differentiating specific from non-specific binding to oligonucleotide microarrays,” Nucleic Acids Research 30, (2002))). NSB can occur under certain experimental conditions (e.g., low ionic strength) between non-target species and immobilized avidin which has a net positive charge at neutral pH. These measurements are made as a function of Cₙ, where larger concentrations in the range 10⁻⁶ to 10⁻¹⁰ are used. Here, both association (i.e., binding) and dissociation curves 76 and 78, respectively, are obtained, as illustrated in FIG. 7. The dissociation curve 78 is generated by flowing buffer solution through the flow cell, and allows determination of k_{d} for non-specific binding. From the determination of the kinetic coefficients (k_{α} and k_{d} which should be relatively constant across concentration of non-specific species), one can then use the modified dual-rate two-compartment model to differentiate between specific and non-specific binding, where the fitting parameters in the model is C₁ (initial target concentration) and C₂ (initial concentration of species that non-specifically bind). Herron et al. (J. N. Herron, S. zumBrunnen, J.-X. Wang, X.-L. Gao, H.-K. Wang, A. H. Terry, and D. A. Christensen, “Planar waveguide biosensors for nucleic acid hybridization reactions,” Proceedings SPIE 3913, 177-184 (2000)) showed that NSB can be virtually eliminated by using a neutravidin monolayer instead of avidin. If it determines that NSB is occurring to the point that NSB kinetics cannot be discriminated from the target kinetics, then one should modify the derivatization procedure to employ neutravidin on silanized surfaces according to Herron et al. (for the Type I embodiment) or biotinylated thiol (D. M. Disley, D. C. Cullen, H. X. You, and C. R. Lowe, “Covalent coupling of immunoglobulin G to self assembled monolayers as a method for immobilizing the interfacial-recognition layer of a surface Plasmon resonance immunosensor,” Biosensors and Bioelectronics 13, 1213-1225 (1998)) (for the Type II embodiment), followed by characterization procedures as described previously.

Measurements are then performed using both the target and background species. The background oligos diffuse into the nanocavities and produce a random background signal. Three sets of measurements are made to determine the detection sensitivity. The first set is as a function of target concentration Cₙ, where the concentration of non-specific oligos is also Cₙ. This situation simulates the conditions for a two-zone sensor array. The second set has target concentration Cₙ, but non-specific concentration 10 Cₙ, thus simulating a 10-zone array. The final set has non-specific concentration of 100 Cₙ. From the two-compartment model, one can then determine the minimum detectable target concentration and the associated number of bound target molecules. It is anticipated that the detection limit is a factor of Mₜₒₜ/η lower for the nanocavity architectures than the waveguide (taking into account the normalization between surface intensity of the evanescent field of the waveguide and the intensity of direct excitation on the quartz reference surface from which Mₜₒₜ was derived, where this normalization factor will be of order 1). These measurements should demonstrate that the nanocavity array has improved background isolation as compared to the planar waveguide (as evidenced by greater ratio between the hybridization signal and background noise). In addition, these studies allow the determination of the hybridization time required to obtain quantitative determination of target concentration, as a function of that target concentration (i.e., lower C_{T} will require longer hybridization times). Because of the background isolation and increased detection sensitivity, it is expected that the necessary hybridization time is at least a factor 1/η shorter with the nanocavity array zones as compared to the waveguide zones.

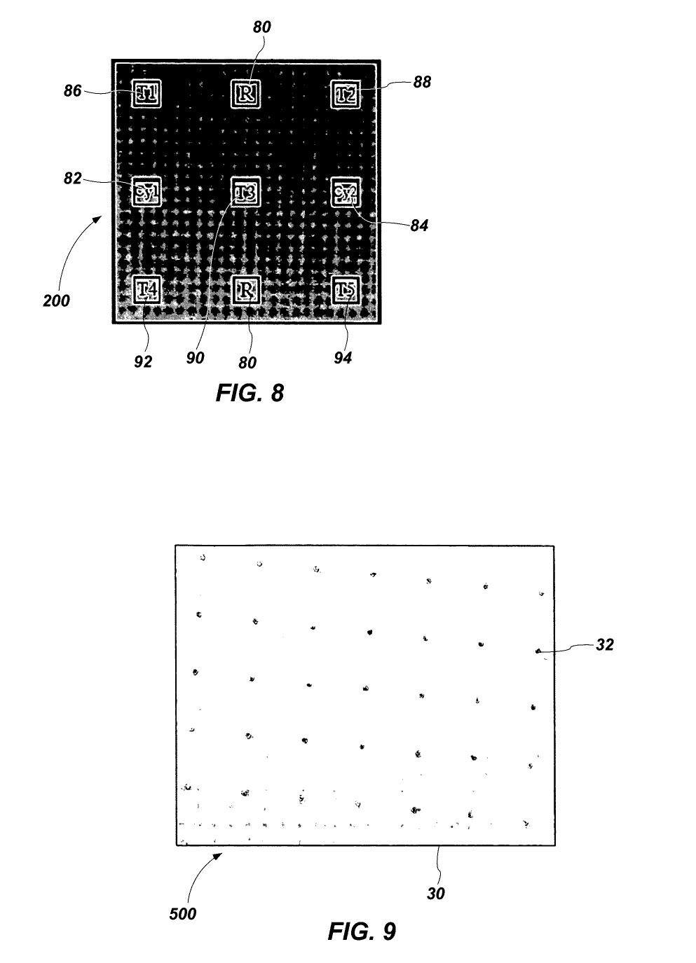

Validation studies. To validate the array system, the 3×3 array 200 (as shown in FIG. 8) is used to screen across multiple synthetic targets (five different 60-mer sequences with varying degrees of overlap, with two sequences differing by only a single base) using five hybridization zones 86, 88, 90, 92 and 94 that are derivatized with complementary probes and the remaining four zones, 80, 80, 82, and 84. The detection zones are placed far enough apart to enable mutual isolation of the detection zones during immobilization of probe molecules. The purpose of using 60-mers is that they are more representative of the sequence length of PCR products in clinical diagnostics setting. Because of the increased oligo length, the hybridization kinetics is slower than in the previous studies due to reduced diffusion. Two of four reference zones are derivatized with Cy-5 labeled probes at different concentrations (to be used as fluorescence intensity references) and the remaining two with anti-T3 (where Cy-5 labeled T3 are introduced in high concentrations in all experiments as a model source of background and NSB), as illustrated in FIG. 8.

In these studies, the five target species in varying concentrations (roughly 10 pM to 1 nM) with T3 at 100 nM concentration are introduced as background. The goal is to study discrimination across the five targets (in terms of obtaining CT for each) in a complex environment where hybridization kinetics varies strongly across hybridization zones due to differing target concentration and where heteroduplex formation occurs. Again, in addition to the quantitative determination of target concentrations, important outcomes are the hybridization time required to make that determination and comparison with a planar waveguide.

These studies determine the ultimate performance of metallic nanocavity arrays as detection zones of a real-time hybridization array. Validation of this platform in situations relevant to clinical diagnostics, in particular, to infectious disease where assay time is critical, is performed. It is anticipated quantitative determination of target concentrations can be made with hybridization kinetics in less than 30 minutes, even in complex environments where the effects of non-specific binding and heteroduplex formation are important. It should also be noted that further refinement of the techniques may be possible by using electric-field enhanced hybridization (R. J. Heaton, A. W. Peterson, and R. M. Georgiadis, “Electrostatic surface plasmon resonance: direct electric field-induced hybridization and denaturation in monolayer nucleic acid films and label-free discrimination of base mismatches,” Proceedings of the National Academy of Sciences 98, 3701-3704 (2001); H.-J. Su, S. Surrey, S. E. McKenzie, P. Fortina, and D. J. Graves, “Kinteics of heterogeneous hybridization on indium tin oxide surfaces with and without an applied potential,” Electrophoresis 23, 1551-1557 (2002)) where it has been shown that hybridization kinetics can be increased (through drift-induced oligo transport to the surface, such that k_{M} in the two-compartment model would increase in value) in addition to improving binding specificity through field reversal. The gold metallic layer upon which the nanocavity array sensing zones are fabricated would lend itself naturally to such a technique.

### Example 4

### Quantitative Study and Comparison of Enhanced Molecular Fluorescence by Periodic Metallic Nanostructure Architectures

The following study is designed to quantitatively compare fluorescence enhancement mechanism and detection sensitivities in complex environments for three periodic metallic nanostructure architectures for real-time hybridization arrays. Each nanostructure arrangement is conducive to real-time measurement of hybridization kinetics, is scalable to a large array format, and may possess sufficient molecular sensitivity to bypass the need for molecular amplification steps required by other methodologies.

### Example 4.1

### Fabrication of Metallic Nanostructure Arrays

To develop nanofabrication methods that are both expedient and repeatable, fabrication of metallic nanostructure arrays are based upon the technique of lift-off, which bypasses the need for a hard mask and metal dry-etching. Dry etching of metals requires very tight process control to produce repeatable results. Lift-off therefore results in significantly increased device yield.

Even though expensive from a manufacturing standpoint, e-beam lithography is the most stable, cost-effective, and flexible nanolithography tool available in an academic environment. Other techniques to fabricate metallic nanostructure arrays include interference lithography (S. C. Lee and S. R. Brueck, “Nanoscale two-dimensional patterning on Si(001) by large-area interferometric lithography and anisotropic wet etching,” Journal of Vacuum Science & Technology B 22, 1949-1952 (2004)), which exposes patterns in photoresist based upon the interference of two or more optical plane waves; nanosphere lithography (W. A. Murray, S. Astilean, and W. L. Barnes, “Transition from localized surface plasmon resonance to extended surface plasmon-polariton as metallic nanoparticles merge to form a periodic hole array,” Physical Review B 69, 165407 (2004)), in which a self-assembled monolayer of small dielectric spheres is used as a mask for deposition/etching steps; and nanoimprint lithography (S. Y. Chou, P. R. Krauss, and P. J. Renstrom “Nanoimprint lithography,” Journal of Vacuum Science & Technology B 14, 4129-4133 (2004)), in which arbitrary patterns can be written using e-beam or focused ion beam lithography on a master, then the master used to stamp out multiple copies (in an analogy to the process used for CD's and DVD's, which are basically a thin metal layer surrounded by dielectric layers).

Three array architecture, including Nanocavity array architecture, Bullseye architecture, Nanoparticle array architecture, are fabricated. For each architecture, individual detection zones which are 75 μm×75 μm in size are fabricated.

Nanocavity array architecture. Nanocavity arrays are produced in 60 nm thick gold films using electron beam lithography followed by a reactive ion etching (RIE) step. The gold layer is deposited on clean quartz substrates by RF-magnetron sputtering followed by a 300 nm silicon-nitride film deposited by plasma enhanced chemical vapor deposition (PECVD). Then a layer of PMMA is spun on for 45 seconds at 4000 rpm and baked to remove the solvent. An identical second coating is applied with an additional baking step thereafter to produce a PMMA layer of total thickness 350 nm. The nanocavity array patterns are drawn on this positive resist using e-beam and the exposed PMMA developed in a solution of MIBK:IPA 1:3 for 70 seconds. The pattern from PMMA is transferred to silicon-nitride using RIE with etching gases of CF₄ and O₂, then continue to transfer the pattern to gold with etching gases Cl₂ and Ar₂. FIG. 9 shows an SEM image 500 of one nanocavity array 30. The array 30 has Λ=1 μm and d=150 nm. In addition, a ^{˜}20 nm layer of SiON (preceded by a thin adhesion layer) is deposited on top of the ^{˜}100 nm gold layer before lift-off. After lift-off, the only exposed gold surfaces are the interior walls of the nanocavities. This modified structure has two advantages. First, because the top and bottom metal interfaces are more nearly symmetric in terms of the effective propagation constants of the SPP modes at each interface 3, there is stronger coupling between these SPP modes, resulting in greater light transmission (L. Martin-Moreno, F. J. Garcia-Vidal, H. J. Lezec, K. M. Pellerin, T. Thio, J. B. Pendry, and T. W. Ebbesen, “Theory of extraordinary optical transmission through subwavelength hole arrays,” Physical Review Letters 86, 1114-1117 (2001)) and therefore, greater intensity enhancement within the nanocavities. In this symmetric situation, the maximum intensity enhancement is estimated to be a factor of 7 (H. J. Lezec and T. Thio, “Diffracted evanescent wave model for enhanced and suppressed optical transmission through subwavelength hole arrays,” Optics Express 12, 3629-3651 (2004)) The second advantage is that the same surface modification chemistry can be shared between the nanocavity and nanoparticle architectures, where selective derivatization of the exposed gold surfaces is performed.

It is well-known (H. J. Lezec and T. Thio, “Diffracted evanescent wave model for enhanced and suppressed optical transmission through subwavelength hole arrays,” Optics Express 12, 3629-3651 (2004)) that the transmission enhancement is maximum for nanocavity spacings Λ slightly less than the excitation wavelength λ, and for nanocavity diameters d^{˜}λ/3. However, because of nanocavity effects, this may not be optimal for fluorescence emission. Therefore, nanocavity diameters ranging from 100 to 250 nm are fabricated. This optimal spacing is designed for normally incident light. In order to minimize transmitted excitation light from producing background signal at the detector (i.e., leakage through the spectral filter), spacings in the 750-850 nm range are used, as this allows collection of fluorescence emission normal to the surface over a ±10° cone half-angle without the collection of transmitted excitation light, which exits at 12-17°. After sample fabrication, linear transmission measurements versus wavelength at normal incidence and versus angle of incidence at a fixed wavelength (633 nm) are performed. These measurements allow for parameter optimizations to maximize transmission and to determine sample-to-sample repeatability.

Bullseye architecture. Nanofabrication of this architecture is quite a bit more involved than the nanocavity sub-arrays. A new process for fabricating this architecture can be based upon e-beam lithography. This process requires accurate alignment (better than about 60 nm) of one e-beam lithography step to another. The first step is to place alignment marks on the substrate using optical lithography. These alignment marks are made using small gold crosses and are oriented at the corners of an 80 μm by 80 μm square, which is approximately the field of view of the e-beam system. The next step is to pattern the 3×3 sub-array of circular corrugations into the quartz substrate with S=25 μm center-to-center spacing. This process occurs via e-beam lithography with positive resist. Before exposure, the e-beam is switched to imaging mode to locate the calibration marks. Once located, a grid can be created on the controlling computer with the alignment marks at the corners. The bullseye sub-array pattern is then exposed using coordinates on this grid with a periodicity 580 nm, which is nearly optimal for an excitation wavelength of 633 nm (T. Thio, K. M. Pellerin, R. A. Linke, H. J. Lezec, and T. W. Ebbeson, “Enhanced light transmission through a single subwavelength aperture,” Optic Letters 26, 1972-1974 (2001)). About 10 annular rings are sufficient to achieve the maximum enhancement. After exposure and development, the quartz substrate is dry etched to a depth of about 40-50 nm, which again is nearly optimal (too deep of an etch renders these annular regions of the gold nearly transparent). From here, the process follows the procedure of fabricating the nanocavity arrays, with the exception of the additional alignment step needed during e-beam lithography to place a nanocavity in the center of each circular corrugation. Lift-off leaves a nanocavity in the center of the bullseye, while the corrugation is automatically produced (or cloned) onto the metal by deposition onto the corrugated substrate. After fabrication, these samples are imaged with SEM to determine the placement of the nanocavity with respect to the center of the bullseye corrugation. Then, linear transmission spectra are taken to determine the transmission enhancement factor and the effect of placement accuracy on the enhancement. Since the effective wavelength of the top-surface SPP mode is roughly λ=633 nm, about 0.1λ accuracy is required. The placement accuracy is verified using rigorous electromagnetic simulation in FEMLAB. After the determination of necessary alignment accuracy, bullseye nanocavity patterns that have acceptable placement are used in specific applications. In the event that the device yield is too low (i.e., less than 25%), the nanocavities of this structure can be milled precisely using dbFIB.

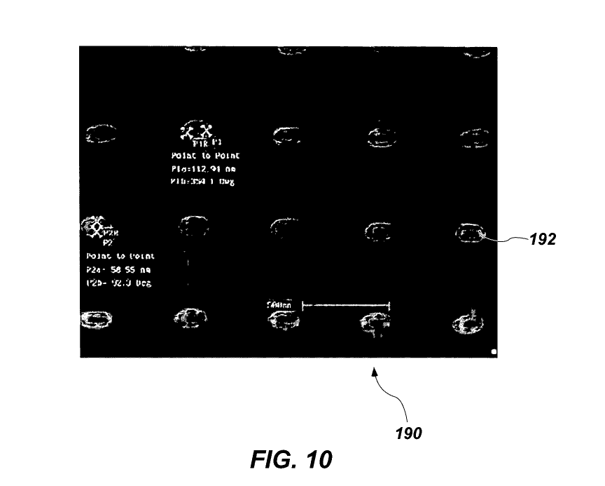

Nanoparticle array architecture. This architecture is the most straightforward to fabricate. A positive e-beam resist (PMMA for example) is spun onto the quartz substrate and baked. The resist is then exposed with the desired nanoparticle array pattern. Developing removes the exposed areas. After deposition of the chromium adhesion and gold layers, the resist is removed during a lift-off process, leaving behind the metallic nanoparticle array. An example nanoparticle array pattern 190 is shown in FIG. 10. The pattern 190 includes particles 192, where the particles are elliptical in shape with 113 nm major axis and 60 nm minor axis, and the thickness is 30 nm.

As opposed to the nanocavity architectures, optical testing is performed on nanoparticle arrays in reflection mode as coupling into the local surface plasmon modes of the nanoparticles occurs via total internal reflection (TIR) from a prism, as illustrated in FIG. 10. The nanoparticles are arranged in a periodic square lattice with Λ^{˜}150-250 nm, which prevents any particle-particle interaction that would shift and dampen the individual nanoparticle resonance (W. A. Murray, S. Astilean, and W. L. Barnes, “Transition from localized surface plasmon resonance to extended surface plasmon-polariton as metallic nanoparticles merge to form a periodic hole array,” Physical Review B 69, 165407 (2004)), while at the same time eliminates any diffraction orders from the incident excitation light as Λ<λ/2. In reflection, the localized surface plasmon resonance of each nanoparticle is indicated by dips (as opposed to peaks in transmission for the nanocavities). Nanoparticle shapes and thicknesses are designed to maximize absorption at 633 nm under TIR illumination, roughly elliptical with 110 nm major axis and 100 nm minor axis and 30 nm thickness.

Improving geometry control. One of the issues that arises during long exposures in electron beam lithography is the stability of the writing beam. Thermal drift of the electron beam may result in slight variation in the positions of nanocavities in an array. This problem worsens with increasing pattern complexity. The effect of nonuniform spacing of nanocavities (i.e., inhomogeneous broadening), for example, is a reduction of the excitation efficiency of surface plasmons, thereby reducing the achievable intensity enhancement factor within the nanocavities. Geometry nonuniformity can be significantly reduced when using “faster” e-beam resists. Fast resists require reduced exposure dosage, and hence the same pattern can be written in less time, but these resists do tend to have reduced resolution. Many different positive and negative resists can be investigated to find a faster resist that retains enough resolution for the patterns.

### Example 4.2

### Selective Derivatization of Nanostructures

The following studies are designed are to develop and characterize selective surface modification procedures for the nanostructure architectures.

Surface modification for measurement of fluorescent enhancement. A passivation procedure for the metal surface has been developed so that the fluorescing monolayer only covers the bottom, quartz, surface of the nanocavities. The passivation procedure follows Prime et al. (K. L. Prime and G. M. Whitesides, “Adsorption of proteins onto surfaces containing end-attached oligo(ethylene oxide): a model system using self-assembled monolayers,” Journal of the American Chemical Society 115, 10714-10721 (1993)): dissolve mPEG-thiol (in powdered form) to a concentration of 1 μmol/L in ethanol; apply solution to gold surfaces for 24 hours in a N₂ filled glove box at room temperature and atmospheric pressure, then rinse in ethanol and dry in N₂; apply labeled avidin solution to form the monolayer. Reference surfaces for this procedure consisted of a smooth gold surface, which after passivation and monolayer coating, did not produce a measurable fluorescence signal in a fluorescence microscope, and a quartz substrate, which after passivation and monolayer coating, produced a fluorescence signal roughly 85% of a coated quartz surface without passivation, which is likely the result of a slight reduction of bound surface concentration.

A similar procedure can be developed for the nanostructure array architectures, where the dielectric surfaces are passivated, thus allowing monolayer formation only on the exposed gold sidewalls of the nanocavities or gold surfaces of the nanoparticles. Dielectric surface passivation is performed using mPEG-silane. Introduction of Cy-5 labeled avidin solution then allows monolayer formation on the exposed gold surfaces. Alternatively, selectively derivatizing the side-walls may also be employed, which follows a procedure developed with biotinylated thiol (D. M. Disley, D. C. Cullen, H. X. You, and C. R. Lowe, “Covalent coupling of immunoglobulin G to self-assembled monolayers as a method for immobilizing the interfacial-recognition layer of a surface plasmon resonance immunosensor,” Biosensors and Bioelectronics 13, 1213-1225 (1998)), which binds strongly to gold surfaces. Cy-5 labeled neutravidin is then be used for the monolayer, where the neutravidin does not undergo a charge-charge interaction with the dielectric surfaces, but interacts strongly with the biotin layer via two of its biotin binding sites. A third alternative is to passivate with mPEG-silane and derivatize the exposed gold surfaces with thiol-conjugated oligos (J. Malicka, I. Gryczynski, and J. R. Lakowicz, “DNA hybridization assays using metal-enhanced fluorescence,” Biochemical and Biophysical Research Communications 306, 213-218 (2003)). These procedures are tested with two reference surfaces, a quartz surface which, after derivatization, should exhibit no measurable fluorescence, and a gold surface which should exhibit strong fluorescence, as measured with a scanning confocal fluorescence microscope.

The new procedures are tested first on the nanoparticle array architecture, as direct imaging by the fluorescence microscope in reflection mode can be used to determine selective derivatization of the nanoparticles with Cy-5/avidin. The other two nanocavity array architectures can be tested next. By using reflection mode, the absence of fluorescence from the top surface can be verified; fluorescence from within the nanocavities can be detected. As further verification, transmission mode can be used to verify that the only detectable fluorescence comes from the nanocavities.

Because these surface modification procedures require multiple steps involving mass transport into and out of nanoscale volumes, it is important that the final derivatized surfaces be fully characterized. One motivating factor behind the characterization studies is the determination of bound avidin surface concentration within the nanocavities or upon the nanoparticles. The characterization methods are described herein in Example 4.

Surface modification for nucleic acid hybridization. The T3 polymerase promotor site is used as a model system for nucleic acid hybridization to determine background isolation and molecular sensitivity across the three architectures. T3 5′-(AATTAACCCTCACTAAAGGG)-3′ and complementary anti-T3 are commercially available, and can be fluorescently labeled with Cy-5. Capture oligonucleotides (anti-T3 for these studies) are immobilized onto the nanostructures. Formation of the avidin monolayer is followed by a solution of 0.1-10.0 μM 5′-biotinylated anti-T3 which self-assembles on top of the avidin-coated surface (J. N. Herron, S. zumBrunnen, J.-X. Wang, X.-L. Gao, H.-K. Wang, A. H. Terry, and D. A. Christensen, “Planar waveguide biosensors for nucleic acid hybridization reactions,” Proceedings SPIE 3913, 177-184 (2000)). Radiolabeled oligonucleotides (anti-T3 for this part of the study) are prepared by end labeling with (³²P)phosphate. For determining the surface concentration of immobilized capture oligo (i.e., anti-T3), 5′-biotinylated oligos are labeled with (a-³²P)ATP using terminal transferase. This enzyme adds (³²P)AMP to the 3′ end of the oligo. A commercial 3′ end labeling kit (Perkin Elmer) is used to perform the reaction. The extra adenosine group is not expected to interfere with binding of the labeled oligo to the immobilized avidin monolayer. Radiolabeling efficiency is determined using a similar procedure as described before with equation (1). The specific activity is determined using a UV-vis spectrophotometer at 260 nm. After self-assembly of the radiolabeled anti-T3 onto the avidin monolayer, the probe surface concentration is determined according to equation (2) using the radioisotope detector. The surface concentration of the respective nanostructure is compared to that obtained for a reference sample. The derivatization procedure for the nanocavities may need to be adjusted as a result of this comparison, either by adjusting concentration of anti-T3 in solution or by changing the adsorption time.

### Example 4.3

### Comparison of Fluorescence Enhancement

Detailed, comparative studies of the total fluorescence enhancement by each of the three architectures are performed. Measurements by Lakowicz′ group (J. Malicka, I. Gryczynski, Z. Gryczynski, and J. R. Lakowicz, “Effects of fluorophore-to-silver distance on the emission of cyanine-dye-labeled oligonucleotides,” Analytical Biochemistry 315, 57-66 (2003)) showed that the fluorescence lifetime of Cy-5 can be reduced from about 1.3 ns to less than 100 ps on nanostructured metallic surfaces, which is one reason why it is necessary to build a system with such small time resolution. In a simplified phenomenological model, the total fluorescence enhancement for all three architectures is given by the product of three factors—Mₜₒₜ=M_{SP}M_{rad}Mᵣₐₜₑ, where M_{SP} is the enhancement factor of the incident intensity due to surface plasmon coupling, M_{rad} is the enhancement in fluorescence due to interaction of the molecular radiative dipole with the metallic nanostructure (L. A. Blanco and F. J. G. do Abajo, “Spontaneous light emission in complex nanostructures,” Physical Review B 69, 205414 (2004); Y. Liu and and S. Blair, “Enhanced fluorescence transduction properties of metallic nanocavity arrays,” submitted to IEEE Journal of Selected Topics in Quantum Electronics (2005)), and Mᵣₐₜₑ is the enhancement factor associated with increase in the radiative transition rate (J. Malicka, I. Gryczynski, Z. Gryczynski, and J. R. Lakowicz, “Effects of fluorophore-to-silver distance on the emission of cyanine-dye-labeled oligonucleotides,” Analytical Biochemistry 315, 57-66 (2003); L. A. Blanco and F. J. G. do Abajo, “Spontaneous light emission in complex nanostructures,” Physical Review B 69, 205414 (2004)). The product M_{yield}=M_{rad}Mᵣₐₜₑ results in an apparent increase in fluorescence yield, even though the actual quantum efficiency may not be greatly increased. The fluorescence quantum efficiency of a fluor is given by

QE=kᵣ/(kᵣ+kₙᵣ)

where kᵣ is the radiative rate and kₙᵣ is the nonradiative rate of de-excitation. In the case where the radiative rate is modified to a new value k′ᵣ, the fluorescence enhancement is given by the factor Mᵣₐₜₑ=QE′/QE, where QE′=k′ᵣ/(k′ᵣ+kₙᵣ). The amount of enhancement therefore depends strongly on the native QE of the fluor. For Cy-5, QE^{˜}28%; therefore, QE can be increased by a maximum of 3.6 times. However, proximity of a fluor to a metal surface can introduce new non-radiative pathways, such as energy transfer to phonons or surface electromagnetic waves. This can also result in a reduction in fluorescence lifetime with comparable reduction in Q, leaving yield unchanged.

Three types of measurements are performed to determine these factors and to compare the three architectures. Measurements of total fluorescence output and photobleaching times are performed as compared to reference surfaces. These measurements allow for estimates of M_{SP} and M_{yield}. Fluorescence lifetime measurements are performed, from which the relative contributions of M_{rad} and Mᵣₐₜₑ can be estimated. Initial measurements are performed using Cy-5 labeled avidin, for which the fluor-to-metal separation is about 5 nm. Because the three enhancement factors may have strong dependence on this separation (as shown in related work for Cy-5 on nanostructured silver surfaces, for which the maximum enhancement occurred at 9 nm separation (J. Malicka, I. Gryczynski, Z. Gryczynski, and J. R. Lakowicz, “Effects of fluorophore-to-silver distance on the emission of cyanine-dye-labeled oligonucleotides,” Analytical Biochemistry 315, 57-66 (2003))), a simple technique is employed to experimentally study this distance dependence using alternating layers of avidin and biotinylated BSA (BBSA). The monolayer sequence starts with avidin:Cy-5; the next step in the sequence is avidin:BBSA:avidin:Cy-5; and so on. At each step, layer thickness is measured by ellipsometry and surface concentration measured by radiolabeling.

For each architecture, a rigorous electromagnetic simulation (using FEMLAB) of the radiative properties of a dipole (using all orientations) is performed, either within a metallic nanocavity or adjacent to a metallic nanoparticle. In order to mimic experimental conditions, the dipole-metal distance is varied from about 5 nm to about 25 nm. Combined with the experimental results, the results of these simulations can provide further insight into the photophysical processes occurring as the result of proximity to the metallic nanostructures. These studies also determine the maximum oligo lengths that can be employed while retaining the benefits of enhanced fluorescence; preliminary studies suggest that 60-base oligo's still maintain significant enhancement (Y. Liu and S. Blair, “Enhanced fluorescence transduction properties of metallic nanocavity arrays,” submitted to IEEE Journal of Selected Topics in Quantum Electronics (2005)) (i.e., greater than half the maximum) within a nanocavity.

Nanocavity array architecture. Previous studies indicate that the intensity enhancement factor due to surface plasmon coupling M_{SP} is about a factor of 2, but with the new symmetric structure described herein, this factor may increase to nearly 7 (H. J. Lezec and T. Thio, “Diffracted evanescent wave model for enhanced and suppressed optical transmission through subwavelength hole arrays,” Optics Express 12, 3629-3651 (2004)). The origin of the excitation enhancement within the nanocavities results from the fact that under the condition of enhanced transmission (Y. Liu and S. Blair, “Fluorescence enhancement from an array of sub-wavelength metal apertures,” Optics Letters 28, 507-509 (2003)), energy is concentrated within the nanocavities. The periodicity of the nanocavities not only supports coupling incident light from free space into surface plasmon modes, but also modifies the propagation properties of the surface plasmon (I. I. Smolyaninov, W. Atia, and C. C. Davis, “Near-field optical microscopy of two-dimensional photonic and plasmonic crystals,” Physical Review B 59, 2454-2460 (1999)) through coherent scattering off the walls of the nanocavities, which results in constructive interference within the nanocavities. Previous studies using random nanocavity arrangements also indicated that the net enhancement of fluorescence yield ranged from about 9 for 150 nm diameter cavities to about 7 for 200 nm diameter cavities. This observation suggests a strong influence of the nanocavity geometry on the emission properties of the fluors, most likely based upon the nanocavity aspect ratio h/d, where h is the height. This effect can be studied experimentally by using nanocavity diameters ranging from 100-250 nm in diameter and comparing with results from simulation. As a result of the geometric dependence, the experimental finding may differ from that of Lakowicz (J. Malicka, I. Gryczynski, Z. Gryczynski, and J. R. Lakowicz, “Effects of fluorophore-to-silver distance on the emission of cyanine-dye-labeled oligonucleotides,” Analytical Biochemistry 315, 57-66 (2003)), for example, where the enhancement effects occurred at a nanostructured planar surface, which is known to result in an increase in radiative rate (A. Wokaun, H.-P. Lutz, A. P. King, U. P. Wild, and R. R. Ernst, “Energy transfer in surface enhanced luminescence,” Journal of Chemical Physics 79, 509-514 (1983)).

In these measurements, fluorescence output from periodic and random nanocavity arrays are compared to the output from a reference surface that consists of a quartz surface with the same surface concentration (as determined by radiolabeling). The output from the nanocavity arrays is normalized to the fill-fraction of the fluors, given by η_{f}=πdh/Λ2 (which is different than the nanocavity fill fraction η=π(d/2)²/^{˜}Λ²) since the fluors cover only the inner walls of the nanocavities. The total fluorescence enhancement factor Mₜₒₜ is given by the ratio of normalized fluorescence output from the periodic array to the reference surface, while the surface plasmon excitation enhancement factor M_{SP} is given by the ratio of fluorescence between the periodic and random array. From these quantities, an apparent increase in fluorescence yield can be determined by the ratio M_{yield}=Mₜₒₜ/M_{SP}, but further measurements can be compared to compare photobleaching times and fluorescence lifetimes across the periodic and random nanocavity array geometries and the reference in order to obtain a better understanding of the influence of the nanocavity geometry on the photophysical processes.

All measurements using three different samples are performed for each geometry. Multiple samples can be fabricated on each substrate to greatly improve the efficiency. The standard deviations from these measurements reflect variations in pattern fabrication, monolayer coating, and optical alignment and detection. With our previous methods, we achieved a standard deviation of less than 10% of the mean. The new fabrication methods and surface modification procedures are expected to reduce the deviation to less than 5%.

Bullseye nanocavity architecture. The main reason why the surface plasmon enhancement factor for the nanocavity arrays is small is due to the fact that the periodicity of the nanocavities themselves is used to couple incident light into surface plasmon modes. The fill fraction of the nanocavities is small (resulting in a weak diffraction grating) in order to keep Λ>λ and to improve background isolation. The bullseye nanocavity geometry breaks these constraints by using a separate, more efficient, structure for grating coupling into surface plasmon modes—corrugated annular rings—which redistributes a much larger fraction of incident light into the nanocavity in the center. Measurements of second-harmonic generation through the bullseye nanocavity compared to a single, bare nanocavity (A. Nahata, R. A. Linke, T. Ishi, and K. Ohashi, “Enhanced nonlinear optical conversion using periodically nanostructured metal films,” Optics Letters 28, 423-425 (2003)) indicate that the surface plasmon intensity enhancement within the bullseye nanocavity M_{SP}^{˜}100. The main disadvantage of the bullseye architecture is that the annular ring structure is significantly larger than the nanocavity, reducing the density of detection sites within a zone.

As before with the nanocavity array architecture, measurements across three samples—a 3×3 bullseye array, and 3×3 nanocavity array with Λ=S=25 μm, and the quartz surface, are compared. The purpose of the 3×3 nanocavity array is to maintain the same fluor fill fraction η_{f}=πdh/S² while isolating surface plasmon excitation enhancement from nanocavity related effects. At 25 μm spacing, which is greater than the surface plasmon attenuation length, these nanocavities do not coherently interact and therefore act as independent cavities. From these measurements, significantly larger excitation enhancement factors M_{SP} is expected with very similar yield enhancements M_{yield}. Because of the annular corrugation surrounding each nanocavity in the bullseye structure, the radiative properties of the fluor could be modified from that of the bare nanocavity, which would affect the radiative efficiency M_{rad}. This can be verified with electromagnetic simulation. Even with the possible modification of M_{rad}, it is anticipated that it is not necessary to repeat the exhaustive studies comparing enhancement effects versus nanocavity diameter d and fluor-to-sidewall separation, thus requiring far fewer bullseye patterns be fabricated.

Nanoparticle architecture. Large surface enhancement effects by metallic nanoparticles have been known for over 20 years (M. Fleischmann, P. J. Hendra, and A. J. McQuillan, “Raman spectra of pyridine adsorbed at a silver electrode,” Chemical Physics Letters 26, 163-166 (1974); H. G. Craighead and A. M. Glass “Optical absorption of small metal particles with adsorbed dye coats,” Optics Letters 6, 248-250 (1981)) owing to the large absorption cross-section associated with the local plasmon resonance of the nanoparticle. The wavelength of the peak absorption due to the local plasmon resonance is determined by the geometry of the nanoparticle. The nanoparticles are elliptical in shape with major axis about 110 nm, minor axis about 100 nm, and thickness about 30 nm, which places the peak of the local plasmon absorption near 633 nm in one linear polarization state (the state of polarization of the excitation light) and a peak in the local plasmon resonance near 670 nm (the peak fluorescence wavelength for Cy-5) for the orthogonal state of polarization. The use of an elliptical nanoparticle can produce a double resonance effect (H. Ditlbacher, N. Felidj, J. R. Krenn, B. Lambprecht, A. Leitner, and F. R. Aussenegg, “Electromagnetic intereaction of fluorophores with designed 2D silver nanoparticle arrays,” Applied Physics B 73, 373 (2001)) to maximize fluorescence output. With the nanoparticle array, since the nanoparticles are non-interacting, there is no way to directly isolate the excitation enhancement factor M_{SP} from M_{yield}; therefore, the comparisons are based upon the quartz reference surface. The measured fluorescence from the nanoparticle array is normalized to the fill fraction of the nanoparticles η=π(d/2)²/Λ² and compared to the reference sample to obtain the total fluorescence enhancement factor. This fill fraction is significantly greater than for the nanocavity array as Λ is much shorter.

For the nanoparticle array, M_{SP} can be estimated from the ratios of photobleaching times and fluorescence lifetimes between the nanoparticle array and reference surface. As with the nanocavity array, these measurements are performed as a function of fluor-particle spacing, but expect to obtain results qualitatively similar to Lakowicz (J. Malicka, I. Gryczynski, Z. Gryczynski, and J. R. Lakowicz, “Effects of fluorophore-to-silver distance on the emission of cyanine-dye-labeled oligonucleotides,” Analytical Biochemistry 315, 57-66 (2003)). Overall, total enhancement factors Mₜₒₜ>10 is anticipated (H. Ditlbacher, N. Felidj, J. R. Krenn, B. Lambprecht, A. Leitner, and F. R. Aussenegg, “Electromagnetic intereaction of fluorophores with designed 2D silver nanoparticle arrays,” Applied Physics B 73, 373 (2001); J. Malicka, I. Gryczynski, and J. R. Lakowicz, “DNA hybridization assays using metal-enhanced fluorescence,” Biochemical and Biophysical Research Communications 306, 213-218 (2003); J. Malicka, I. Gryczynski, Z. Gryczynski, and J. R. Lakowicz “Effects of fluorophore-to-silver distance on the emission of cyanine-dye-labeled oligonucleotides,” Analytical Biochemistry 315, 57-66 (2003)), with the majority of the enhancement due to M_{SP}. The total fluorescence enhancement is anticipated to be more sensitive to separation for the nanoparticles than for the nanocavity architectures.

The surface plasmon enhancement factor M_{SP} represents an additional enhancement over the yield enhancement given by M_{yield}=M_{rad}Mᵣₐₜₑ. This additional factor is important in situations where fluorescence transduction is not photobleaching limited. One situation is in the use of quantum dot fluorescence labels (M. B. Jr., M. Moronne, P. Gin, S. Weiss, and A. P. Alivisatos, “Semiconductor nanocrystals as fluorescent biological labels,” Science 281, 2013-2016 (1998); W. C. W. Chan and S. Nie “Quantum dot bioconjugates for ultrasensitive nonisotropic detection,” Science 281, 2016-2018 (1998).), which do not significantly photobleach. With metallic nanostructure arrays comprising a single zone, we obtain the practical advantages of an additional fluorescence signal increase by the factor M_{SP}M_{yield} for the same number of fluorescing molecules, or a reduction in the number of molecules by the factor M_{SP}M_{yield} with the same fluorescence level, as compared to direct excitation on a quartz substrate with the same zone area. The other situation is that of simultaneous transduction of multi-zone sensing arrays, in which the incidence light is divided equally among all zones. The practical advantage, then, even in the case of fluors that photobleach, is that spreading the light across MSP zones will result in M_{yield} times more output from each zone with the same bleaching time, as compared to a single zone on a quartz substrate with the same number of molecules per zone. One could also excite M_{SP}M_{yield} zones with the same fluorescence output from each zone as the quartz substrate, but with a much longer time to photobleach. These factors do not take into account the other significant advantage of the nanostructure array architectures, that of background isolation, which further improves these scalings by at least another order of magnitude.

### Example 4.4

### Comparison of Sensitivity in Real-Time Hybridization

The performance advantages of the nanostructure architectures are quantified through the study of real-time hybridization kinetics on a single zone using fluorescence transduction in the presence of varying concentrations of background species. Instead of using RNA as the target species (as would be the case in direct expression analysis), end-labeled single-stranded DNA (i.e., an oligonucleotide of 20-base length) is used. The reason for this is that DNA is significantly more robust and requires fewer precautions in sample handling and storage, therefore simplifying experimental procedures. These results are highly relevant to the direct analysis of expressed RNA, which can also be performed using the structures as described herein. The T3 polymerase promotor site is used as a model system; T3 and complementary anti-T3 are commercially available, and can be fluorescently labeled with Cy-5. Capture oligonucleotides (anti-T3 for these studies) are selectively immobilized as described previously. Sample solution containing fluorescently labeled target and non-target species are introduced to the surface using a flow cell (see FIG. 4), which resides on the top surface of the nanostructure samples. A kinetic curve is analyzed using the two-compartment model as described previously. These parameters are illustrated in FIG. 6. The purpose for using the two-compartment model is that, through the fitting constant k_{M}, the effect of mass transport to the sparse array of detection sites can be determined and compared to a planar waveguide sensing modality in which the fill fraction is 100%. In order to differentiate the effects of non-specific binding, the two-compartment model is modified to describe the binding of two species to the surface with association constants kₐ₁ and kₐ₂. In this case, two bound concentrations B1(t) and B2(t) are obtained, and the density of available binding sites are given by RT-B1(t)-B2(t). The effects of dissociation for the non-specific species may also be incorporated. It is not be noted that the ultimate goal for expression arrays is to use the two-compartment model to analyze the kinetic curve at each hybridization zone to obtain the unknown concentration C_{T} of the desired target in solution.

Before the real-time hybridization experiments are performed, a calibration between bound surface concentration and fluorescence intensity is performed. Using radiolabeling as described previously, the bound probe concentration can be obtained. The same surface modification procedures with Cy-5 end-labeled anti-T3 (with labeling ratio determined by UV-vis absorption) can be performed to allow direct relation between measured fluorescence intensity and bound concentration. This relationship can then be used in conjunction with the two-compartment model to determine the detection limits in terms of the number of bound target molecules.

Determination of detection sensitivity—nanocavity architectures. In this section, fluorescence isolation from unbound species is verified, and the detection sensitivity is determined taking into account non-specific binding for the periodic nanocavity array and 3×3 bullseye array. Because detectable fluorescence can only be produced from within a nanocavity, random variation in fluorescence from non-target molecules only occurs when those molecules randomly diffuse into and out of the nanocavity (although some fraction may non-specifically bind). The nanocavity surface area represents a fraction η of the total zone area (η^{˜}1-4% for the nanocavity arrays and η^{˜}0.01% for the bullseye arrays), so that the background signal from unbound species should be less by a factor of approximately 1/η than in other washless, surface selective fluorescence sensors such as a planar waveguide or fluorescence-SPR where the sensing surface represents 100% of the zone area.

Because the transduction area in the nanocavity architectures is so small, diffusion of the target molecules into the sensing regions may be slower than if the transduction area were 100% of the sensing area. The first step is to study hybridization kinetics of the labeled target as a function of target concentration in solution, as compared to a planar waveguide. Target oligos (T3), labeled at the 5′ end with Cy-5 dye are prepared in solution with a concentration Cₙ, where n is the trial number. Typical Molar concentrations range from 10⁻⁸ to 10⁻¹². When introduced into the flow cell, T3 specifically binds to probe oligos on the capture monolayer and form hybridized DNA.

The hybridization kinetic curve 74 is measured for each Cₙ through the time dependence of the fluorescence excited by light intensity within each nanocavity (as in FIG. 5). Both association 76 (i.e., binding) and dissociation 78 curves are obtained, as illustrated in FIG. 7. The dissociation curve 78 are generated by flowing buffer solution through the flow cell, and allows determination of k_{d} (which will essentially be zero for specific binding, but non-zero for non-specific binding, as described in the next paragraph). By comparing the kinetic curves between the two nanocavity architectures and the waveguide, with greatly different fill fractions, the increase in diffusion time can be determined via the parameter k_{M}.

The next step is to perform the same measurements using a second labeled sequence of the same length as T3/anti-T3 to determine the kinetic coefficients in the two-compartment model for non-specific binding. The sequence of these “background” oligos is chosen so as not to specifically bind to either the target or probe molecules. These background oligos diffuse into the nanocavities and produce a random background signal, which could mask the kinetic curve produced by bound species, and may also non-specifically bind, which produces a signal that mimicks the kinetics of the target species (but with a different rate and equilibrium value (H. Dai, M. Meyer, S. Stepaniants, M. Ziman, and R. Stooughton, “Use of hybridization kinetics for differentiating specific from non-specific binding to oligonucleotide microarrays,” Nucleic Acids Research 30, (2002))). As before, these measurements are made as a function of Cₙ, where larger concentrations in the range 10⁻⁶ to 10⁻¹⁰ are used. From the determination of the kinetic coefficients (kₐ₂ and k_{d2}, which should be relatively constant across concentration of non-specific species), the modified dual-rate two-compartment model can then be used to differentiate between specific and non-specific binding, where the fitting parameters in the model are C₁ and C₂.

Three sets of measurements are made to determine the detection sensitivity. The first set is as a function of target concentration Cₙ, where the concentration of non-specific oligos is also Cₙ. This situation simulates the conditions for a two-zone sensor array. The second set has target concentration Cₙ, but non-specific concentration 10 Cₙ, thus simulating a 10-zone array. The final set has a non-specific concentration of 100 Cₙ. From the two-compartment model, the minimum detectable target concentration and the associated number of bound target molecules can be determined. It is anticipated that the detection limit will be a factor of Mₜₒₜ/η lower for the nanocavity architectures than the waveguide (taking into account the normalization between surface intensity of the evanescent field of the waveguide and the intensity of direct excitation on the quartz reference surface from which Mₜₒₜ was derived, where this normalization factor will be of order 1).

Determination of detection sensitivity—nanoparticle architecture. Estimation of the expected signal to background ratio in the nanoparticle architecture is more involved as fluorescence contribution from non-target species can occur either near the nanoparticle surface or between the nanoparticles. The excitation geometry that minimizes contribution from species residing between nanoparticles is shown in FIG. 11a, in which the local plasmon resonance of the nanoparticles is excited via the evanescent wave of a prism. These mean that only unbound species that randomly diffuse to within about 100 nm of the surface produce a contribution. The signal to background ratio of the nanoparticle arrays is given by Mₜₒₜ/(ηMₜₒₜ+(1−η)); therefore, for very large total fluorescence enhancement factors Mₜₒₜ>>(1−η)/η, the background isolation scales the same as the nanocavity architectures. The same experimental procedures used for the nanocavity architectures are repeated for the nanoparticle architecture.

The detection sensitivities are critically compared across the three architectures. As discussed previously, the nanocavity architectures are expected to have the greatest background isolation, with the bullseye having the greatest signal to background ratio due to its large surface plasmon enhancement and very low fill fraction. These bullseye structures are the most promising architecture for the implementation of very high sensitivity detection that can operate with unbiased real populations, as the large surface plasmon intensity enhancement allows efficient scaling to large arrays, where each zone retains the benefit of yield enhancement given by M_{yield}^{˜}10.

Two additional sets of studies can also be performed: 1) single base-pair mismatch discrimination, which is performed in the same manner as the non-specific binding studies, except that the temperature of the nanostructure array is elevated to near the heteroduplex melting temperature, and 2) binding studies using target and non-target species of 60-base length (where the bottom 20 bases of the target are complementary to anti-T3) in order to provide additional relevant results to situations encountered in expression analysis.

### Example 5

Referring to FIG. 11b, an apparatus and technique are illustrated to characterize the fluorescence emission. The apparatus and technique can characterize the fluorescence within single nanometric apertures. For purposes of description, a single nanocavity with a diameter of 120 nm is formed in a gold film with a thickness of 200 nm. This nanoaperture structure offers a reliable test bench and can be reproducibly fabricated with robust techniques. Genet, C.; Ebbesen, T. W. Light in Tiny Holes. Nature 2007, 445, 39-46. Lenne, P. F.; Rigneault, H.; Marguet, D.; Wenger, J. Fluorescence Fluctuations Analysis in Nanoapertures: Physical Concepts and Biological Applications. Histochem. Cell Biol. 2008, 130, 795805. In addition, it has been proven to be a sensitive platform to study enhanced fluorescence emission of single molecules. Gerard, D.; Wenger, J.; Bonod, N.; Popov, E.; Rigneault, H.; Mandavi, F.; Blair, S.; Dintinger, J.; Ebbesen, T. W. Nanoaperture-Enhanced Fluorescence: Towards Higher Detection Rates with Plasmonic Metals. Phys. Rev. B 2008, 77, 045413. Wenger, J.; Gerard, D.; Bonod, N.; Popov, E.; Rigneault, H.; Dintinger, J.; Mahboub, 0.; Ebbesen, T. W. Emission and Excitation Contributions to Enhanced Single Molecule Fluorescence by Gold Nanometric Apertures. Opt. Express 2008, 76, 3008-3020. The influence of different adhesion layers was investigated, namely: 5 nm of chromium or titanium, 10 nm of titanium, and 10 nm of titanium oxide (TiC2) or chromium oxide (Cr203). The aperture diameter was chosen to yield the maximum fluorescence enhancement at 633 nm excitation. For each sample, the fluorescence emission of Alexa Fluor 647 molecules diffusing within the structure was characterized. Wenger, J.; Gerard, D.; Bonod, N.; Popov, E.; Rigneault, H.; Dintinger, J.; Mahboub, 0.; Ebbesen, T. W. Emission and Excitation Contributions to Enhanced Single Molecule Fluorescence by Gold Nanometric Apertures. Opt. Express 2008, 76, 3008-3020. This method relies on a combination of fluorescence correlation spectroscopy (FCS) with fluorescence time-correlated lifetime measurements. It specifically allows one to detail the influence of the nanostructure on the molecular emission and quantifies the respective weights of excitation and emission contributions to the observed enhanced fluorescence.

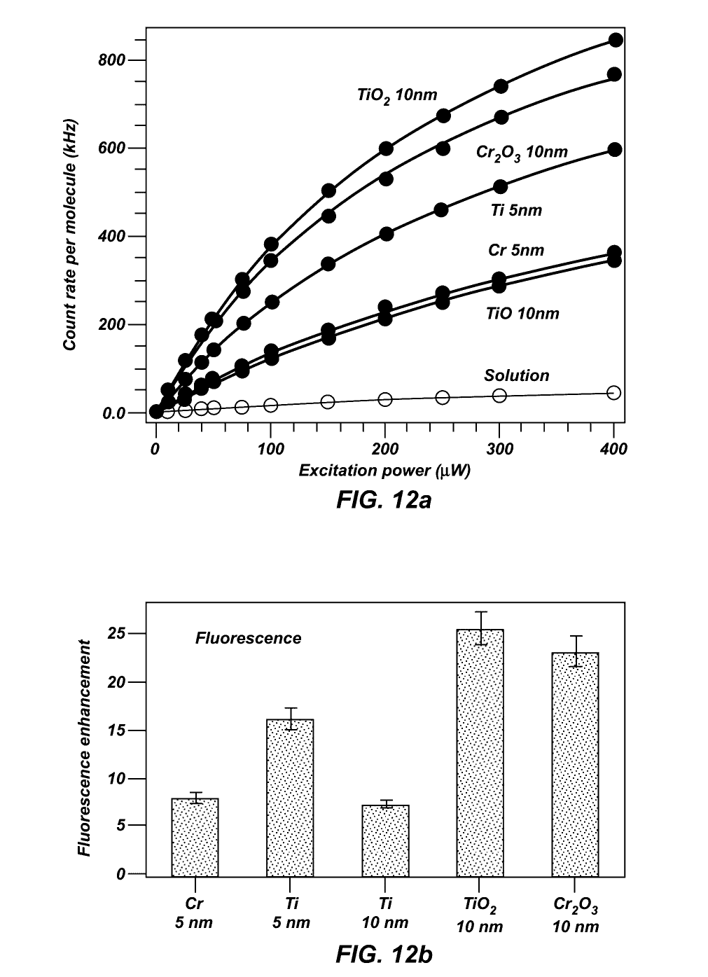

A set of FCS measurements were performed while increasing the excitation power from 10 to 400 W (the upper limit was set to avoid damaging the sample and photobleaching the dyes). Each FCS correlation function was analyzed to reliably measure the fluorescence count rate per molecule (CRM), as described below. For each excitation power, special care is taken to characterize the level of background noise and the dark state amplitude. Results are summarized in FIG. 12a, in the case of an open solution and in the case of 120 nm apertures with different adhesion layers. It is apparent from this raw data that the nature of the adhesion layer has a dramatic impact on the fluorescence emission. To make this effect appear even clearer, we model this data with the expression of the fluorescence rate given by CRM=A(Ie)/(1+Ie,/Is), where Ie is the excitation intensity, Is the saturation intensity, and A is a constant proportional to the molecular absorption cross section, quantum yield, and setup collection efficiency. Zander, C., Enderlein, J., Keller, R. A., Eds. Single-Molecule Detection in Solution: Methods and Applications; Wiley-VCH: Berlin/New York, 2002. The fitting parameters are summarized in Table 1 and are used hereafter to compare the different adhesion layers.

TABLE 1
Refractive Index (n) and Extinction Coefficient (k) of
the Different Materials Used Here at 633 nm Wavelength,
Jiao, X.; Goeckeritz, J; Blair, S.; Oldham, M. Localization
of Near-Field Resonances in Bowtie Antennae: Influence
of Adhesion Layers. Plasmonics 2009, 4, 37-50.

| material | Cr | Ti | Ti | TiO2 | Cr2O3 | sol |
|---|---|---|---|---|---|---|
| thickness (nm) | 5 | 5 | 10 | 10 | 10 | |
| n | 3.54 | 2.15 | 2.15 | 1.97 | 2.45 | |
| k | 4.36 | 2.92 | 2.92 | 0 | 0.54 | |
| A (kHz/μW) | 1.6 | 3.2 | 1.4 | 5.1 | 4.6 | 0.2 |
| Is(μW) | 480 | 320 | 600 | 280 | 270 | 435 |
| τ(ns) | 0.41 | 0.40 | 0.36 | 0.40 | 0.40 | 0.88 |

The table also presents the fitting parameters A, Is, for the experimental data in FIG. 3a and the fluorescence lifetime τ in FIG. 4a.

FIG. 12b displays the fluorescence rate enhancement for the different adhesion layers found in the regime below saturation. This factor is defined as the ratio of the detected fluorescence rate per molecule in the aperture with respect to the CRM in open solution taken at the limit of low excitation ie—0. Practically, it corresponds to the ratio of the A parameters derived from the interpolation of the data curves in FIG. 12a, which are given in Table 1.

A striking 25-fold fluorescence enhancement is found for a 10 nm TiO₂ layer and is the highest gain reported to date for Alexa Fluor 647 molecules in a nanoaperture. This value has to be compared to the 7.2 enhancement found instead for a 10 nm Ti layer. Although the same preparation procedures and experimental setup are used, the net difference between the fluorescence signal per emitter can be as large as 3.5 fold. At least seven different apertures are tested for each adhesion layer, with variation in fluorescence count rate between 6 and 8%, which takes into account effects such as dispersion in aperture diameter. The error bars displayed in FIG. 12b indicate the standard deviations of our measurements.

Metallic adhesion layers, such as Cr and Ti, are shown to yield lower overall fluorescence enhancements and greater saturation intensities than dielectric layers. This can be understood as a stronger damping of the plasmonic resonance due to an increased absorption in the adhesion layer and corroborates the conclusion drawn in Jiao, X. (Localization of Near-Field Resonances in Bowtie Antennae: Influence of Adhesion Layers). The negative effect of losses is confirmed by the lower enhancement found for chromium compared to that for titanium at a fixed thickness (chromium's absorption is about three times that of titanium at 633 nm; see Table 1) and by the fact that the fluorescence enhancement increases when the adhesion layer thickness decreases. Last, the damping effect of absorption is also exhibited for dielectrics, with a higher enhancement for titanium oxide (which shows negligible absorption in the visible range) than for chromium oxide (which has some residual absorption at 633 nm). Almost every experimental study of plasmonic nanoantennas skips the issue of the adhesion layer choice and design, while we clearly show here that it has a dramatic influence.

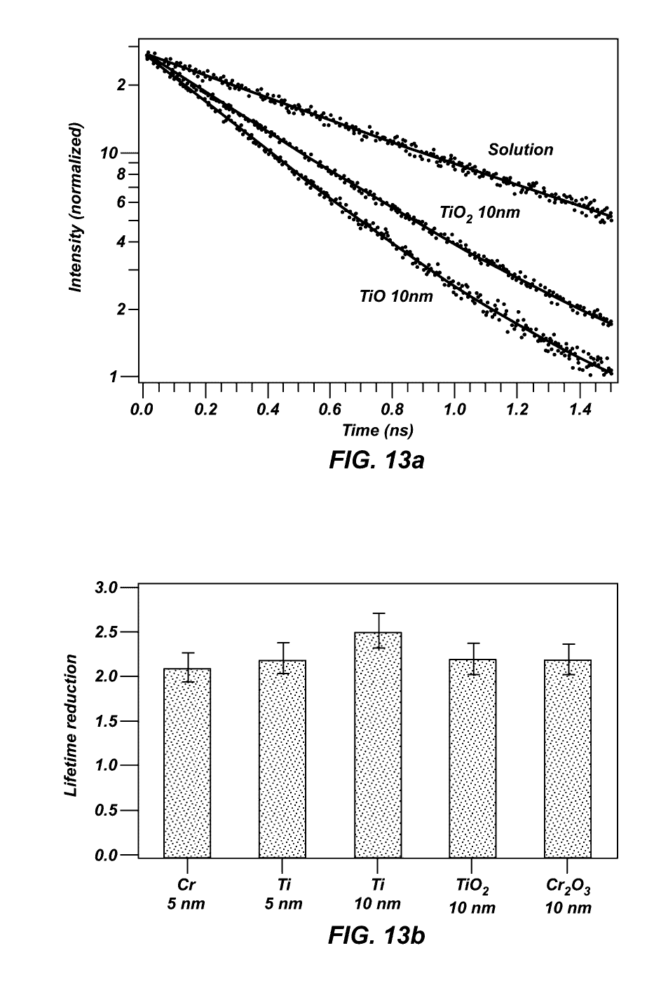

To complete the FCS measurements and fully characterize the fluorescence enhancement phenomenon, we conduct time-correlated single photon counting (TCSPC) experiments to monitor the fluorescence lifetime alteration inside the nanoapertures. These experiments are performed for the same nanoaperture sample and the Alexa Fluor 647 solution. FIG. 13a shows the fluorescence decay curves for molecules in open solution and in single 120 nm apertures with 10 nm Ti or TiO₂ adhesion layer. The other adhesion layers used in this study resulted in traces nearly identical to the one of the TiO₂ case and are not represented to ease viewing the graphs. The fluorescence decay rate is measured by fitting the data using a single exponential decay model convolved by the calibrated instrument response function. Wenger, J.; Gerard, D.; Bonod, N.; Popov, E.; Rigneault, H.; Dintinger, J.; Mahboub, 0.; Ebbesen, T. W. Emission and Excitation Contributions to Enhanced Single Molecule Fluorescence by Gold Nanometric Apertures. Opt. Express 2008, 76, 3008-3020. This yields the fluorescence lifetime reduction normalized to the open solution case displayed in FIG. 13b. Interestingly, this factor appears almost independent of the adhesion layer, showing that the near-field molecular decay routes in the nanoaperture are dominated by the gold structure, not by the adhesion layer. However, the coupling of the fluorescence radiation to the far-field is affected by the adhesion layer, as shown on FIG. 12a and discussed hereafter.

Different effects can lead to an enhancement of the fluorescence signal of a single molecule: (i) local increase in the excitation intensity, (ii) increases in the emitter's radiative rate and quantum efficiency, and (iii) modification of the emitter's radiation pattern, giving a higher emission directionality toward the detectors. Determining the relative influence of these effects is a delicate task, which can be addressed by combining FCS with fluorescence lifetime measurements. As discussed in the Methods section, the gain in emission, ηem, is derived from the value of the fluorescence enhancement in the saturation regime, which we obtain from the asymptotic interpolation of the data points in FIG. 3a at the limit where ie→∞. The gain in excitation intensity, ηexc equals the fluorescence enhancement in the low excitation regime divided by the ratio of the emission gain by the total decay rate alteration:

ηexc=ηF/(ηem/ηtot).

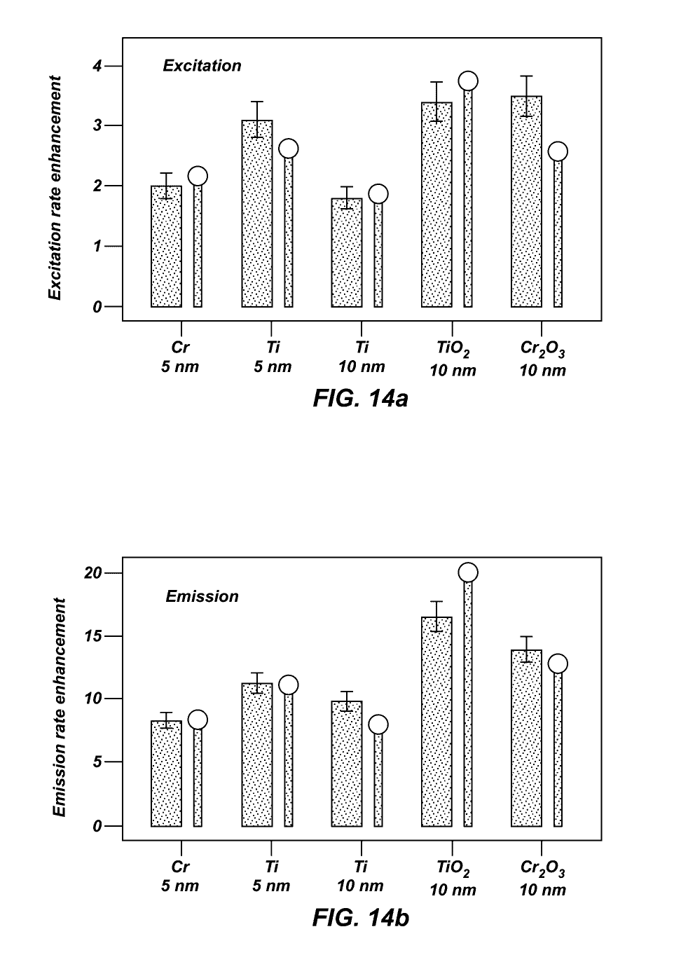

This procedure yields the experimental estimates of the excitation and emission gains for the different adhesion layers displayed in FIGS. 14a and b. The gain in excitation intensity can be understood as a better coupling of energy inside the nanostructure, while the gain in emission has to be related to a better outcoupling of the energy stored in the dipole's near-field to the detected far-field radiation. Due to the small Stokes shift between the excitation and emission wavelengths of Alexa Fluor 647, the reciprocity theorem holds and, consequently, both excitation and emission phenomena influence the overall gain in fluorescence and show strong variations with the nature of the adhesion layer used. Absorption in metallic adhesion layers yields lower gains than dielectric layers for both excitation and emission. These data confirm that any increase in absorption losses due to the material's properties or an increased thickness results in a damping of the near- to far-field coupling at the nanoaperture.

To supplement this experimental evidence, we conduct a numerical analysis based on the finite element method, as discussed below. This method allows one to separately compute the enhancements in excitation intensity and emission rate, which both contribute to the overall gain in the fluorescence signal. The increase of the excitation intensity inside the aperture is computed from the average intensity measured with and without the nanostructure at a plane located 20 nm inside the aperture. For the emission calculations, we compute the radiative emission through the glass side by integrating the z-component of Poynting's vector across a plane located 20 nm below the metal surface for single dipoles located in different positions and orientations inside the aperture, as discussed below. Results of numerical simulations for excitation and emission are displayed in FIGS. 14a and b for the different adhesion layers (empty markers). They are remarkably consistent with the experimental data and confirm the crucial influence of the adhesion layer on both excitation and emission gains in the nanostructure. Interestingly, this method allows one to predict the maximum fluorescence enhancement for gold only with no adhesion layer. We obtain an optimum fluorescence enhancement of 28 for Alexa Fluor 647 molecules, which is about 10% higher than with the 10 nm TiO₂ layer. This confirms that titanium oxide is the material of choice for practical plasmonic enhancement applications.

We experimentally and numerically study the influence of the adhesion layer commonly used to ensure firm contact between a gold film and underlying glass substrate in plasmonic nanoantennas. A single aperture milled in the metal film with 120 nm diameter forms a reliable structure to investigate the effects of the adhesion layer on the fluorescence enhancement of single molecules. Although the same preparation procedures and experimental setup are used, we show that the nature of the adhesion layer (permittivity and thickness) has a dramatic impact on the fluorescence signal per molecule with a difference up to a factor of 4. By combining FCS and fluorescence lifetime measurements, we detail the respective contributions of excitation and emission gains to the observed enhanced fluorescence. Any increase in the absorption losses due to the adhesion layer material's properties or increased thickness is shown to yield lower enhancements, which we relate to a damping of the near- to far-field coupling at the nanoaperture. The experimental data are sustained by numerical simulations using finite element method. To our knowledge, this is the first experimental report of the strong dependence of the fluorescence gain on the nature of the adhesion layer. Clearly, one has to consider the role of the adhesion layer while designing nanoantennas for high-efficiency single-molecule analysis based on either fluorescence or Raman scattering, 10 nm of titanium oxide being the optimal choice based upon our study.

### Methods

Nanoaperture Fabrication. All metal and dielectric films are deposited using reactive DC magnetron sputtering in the same chamber. The gold film thickness of 200 nm was chosen to be optically opaque and isolate the molecules diffusing in the aperture from the pool of molecules lying above the structure. Adhesion between the gold film and the 150 μm thick glass substrate is ensured by a layer of 5 nm of chromium or titanium, 10 nm of titanium, or 10 nm of titanium oxide (TiO₂) or chromium oxide (Cr₂O₃). The oxides' adhesion layers are deposited using the same metal targets, but under partial oxygen pressure. Thickness control is maintained by a cluster of piezoelectric monitors. We relied on calibration of the deposition process using quartz crystal monitors and test samples. Therefore, we estimate no more than 20% error in the adhesion layer thickness. Similar adhesion properties were found for these different layers. Last, circular apertures of 120 nm diameter are milled by focused ion beam (FEI Strata DB235). We checked that the metal film roughness remained similar for all adhesion layers used. The roughness was measured to 1.5 nm.

Fluorescence Count Rate per Molecule Calibration. In order to get an accurate understanding of the fluorescence emission in the nanostructure and investigate the influence of the adhesion layer, it is crucial to quantify the fluorescence count rate per molecule CRM, which requires the knowledge of the actual number of emitters, N, contributing to the global fluorescence signal. This issue is addressed via fluorescence correlation spectroscopy (FCS). Zander, C., Enderlein, J., Keller, R. A., Eds. Single-Molecule Detection in Solution: Methods and Applications; Wiley-VCH: Berlin/New York, 2002. In FCS, the temporal fluctuations, F(t), of the fluorescence signal are recorded, and the temporal correlation of this signal is computed g(2)(τ)={F(t)×F(t+τ)}/{F(τ)}2, where τ is the delay (lag) time, and { } is for time averaging. Analysis of the correlation function provides a measure for the number of molecules, N, needed to compute the count rate per molecule CRM={F}/N. Gerard, D.; Wenger, J.; Bonod, N.;

Popov, E.; Rigneault, H.; Mandavi, F.; Blair, S.; Dintinger, J.; Ebbesen, T. W. Nanoaperture-Enhanced Fluorescence: Towards Higher Detection Rates with Plasmonic Metals. Phys. Rev. B 2008, 77, 045413. Wenger, J.; Gerard, D.; Bonod, N.; Popov, E.; Rigneault, H.; Dintinger, J.; Mahboub, 0.; Ebbesen, T. W. Emission and Excitation Contributions to Enhanced Single Molecule Fluorescence by Gold Nanometric Apertures. Opt. Express 2008, 76, 3008-3020. We point out that, as a consequence of the stochastic nature of the FCS technique, all the presented fluorescence data are spatially averaged over all the possible molecule orientations and positions inside the detection volume.

Excitation and Emission Gains Characterization. To unravel the origins of the fluorescence enhancement near a photonic structure, we have developed a specific experimental procedure which has already been applied to the case of fluorescence alteration by gold nanometric apertures. Wenger, J.; Gerard, D.; Bonod, N.; Popov, E.; Rigneault, H.; Dintinger, J.; Mahboub, 0.; Ebbesen, T. W. Emission and Excitation Contributions to Enhanced Single Molecule Fluorescence by Gold Nanometric Apertures. Opt. Express 2008, 76, 3008-3020. This procedure can be summarized as follows: the fluorescence rates per molecule CRM are measured for increasing excitation powers in open solution and in the case of a nanoaperture. The resulting data points are fitted according to the model CRM A(Ie)/(1+Ie/Is), where Ie is the excitation intensity, Is the saturation intensity, and A is a constant proportional to the molecular absorption cross section, quantum yield, and setup collection efficiency. Zander, C., Enderlein, J., Keller, R. A., Eds. Single-Molecule Detection in Solution: Methods and Applications; Wiley-VCH: Berlin/New York, 2002. We deduce from the fits the fluorescence enhancements at the two extreme cases below saturation Ie<<Is and at saturation Ie>>Is. In the saturation regime, the fluorescence rate enhancement is determined only by the gain in emission, 1 lsm. In the low excitation regime, Ie<<Is, the fluorescence enhancement, if is proportional to the gains in emission, ηem, and local excitation intensity, ηexc, and inversely proportional to the gain in total fluorescence decay rate, ηtot: ηF=ηemηexc/ηtot. Using supplementary fluorescence lifetime measurements to determine the alteration in the total fluorescence decay rate, r, it is therefore possible to extract the gain in local excitation intensity from the fluorescence enhancement in the low excitation regime. This unambiguously separates the excitation and emission contributions to the total fluorescence enhancement and is used here to investigate the influence of the adhesion layer on the fluorescence enhancement in single nanoapertures.

Experimental Setup. A comprehensive description of our experimental setup has been presented before. Wenger, J.; Gerard, D.; Bonod, N.; Popov, E.; Rigneault, H.; Dintinger, J.; Mahboub, 0.; Ebbesen, T. W. Emission and Excitation Contributions to Enhanced Single Molecule Fluorescence by Gold Nanometric Apertures. Opt. Express 2008, 76, 3008-3020. Briefly, it is based on a confocal inverted microscope with a NA=1.2 water-immersion objective, allowing single aperture studies (FIG. 2a). For all experiments reported here, we use an aqueous solution of Alexa Fluor 647 fluorescent molecules (A647, Invitrogen, Carlsbad, Calif.) deposited on top of the sample with micromolar concentration. These molecules are constantly diffusing in and out of the aperture, thereby limiting photobleaching. For FCS measurements, the excitation source is a CW He-Ne laser operating at 633 nm. For lifetime measurements, the excitation source is a picosecond laser diode operating at 636 nm (PicoQuant LDH-P-635). A single-mode optical fiber ensures a perfect spatial overlap between the pulsed laser diode and the CW laser. Accurate positioning of the nanoaperture at the laser focus spot is obtained with a multiaxis piezoelectric stage. Single photon detection is performed by avalanche photodiodes with 670±20 nm fluorescence band-pass filters. For FCS, the fluorescence intensity temporal fluctuations are analyzed with a ALV6000 hardware correlator. Each individual FCS measurement is obtained by averaging 10 runs of 10 s duration. For fluorescence lifetime measurements, the photodiode output is coupled to a fast time-correlated single photon counting module (PicoQuant PicoHarp 300).

Numerical Simulations. Numerical analysis is based on the finite element method using COMSOL Multiphysics version 3.3. Mandavi, F.; Liu, Y.; Blair, S. Modeling Fluorescence Enhancement from Metallic Nanocavities. Plasmonics 2007, 2, 129-142. The model considers a computational space of 1.0×1.0×1.1 μm3, with radiation boundary conditions on all faces. A glass substrate is put underneath a 200 nm thick layer of gold plus a 5 or 10 nm adhesion layer, the upper region being water. Gold dielectric properties are incorporated as measured by spectroscopic ellipsometry. Jiao, X.; Goeckeritz, J.; Blair, S.; Oldham, M. Localization of Near-Field Resonances in Bowtie Antennae: Influence of Adhesion Layers. Plasmonics 2009, 4, 37-50. Mandavi, F.; Liu, Y.; Blair, S. Modeling Fluorescence Enhancement from Metallic Nanocavities. Plasmonics 2007, 2, 129-14. A single 120 nm aperture is placed in the center of the metal. To estimate the increase of the excitation intensity inside the aperture, a plane wave at 633 nm is launched incoming from the glass side. Electromagnetic intensity is measured and averaged over the plane 20 nm inside the aperture. This result is then normalized by the integrated intensity with no metal layer. For the emission calculations, a 1 nm dipole is positioned at various locations inside the aperture. Eleven horizontal planes 20 nm apart are considered in the aperture, the very first and very last planes being 5 nm inside the structure. In each horizontal plane, 37 dipole positions are taken. At each point a dipole emitting at 670 nm is aligned along X, Y, and Z directions. The radiative emission through the glass side is estimated by integrating the z-component of Poynting's vector across a plane located 20 nm below the metal surface. This averaged power is then scaled by a fixed normalization factor to fit the experimental emission gain for the chromium adhesion layer.

Although the description included herewith contains many specifics, these should not be construed as limiting the scope of the invention, but merely as providing illustrations of some of the presently preferred embodiments. Similarly, other embodiments of the invention may be devised which do not depart from the spirit or scope of the invention. Features from different embodiments may be employed in combination. The scope of the invention is, therefore, indicated and limited only by the appended claims and their legal equivalents, rather than by the foregoing description. All additions, deletions and modifications to the invention as disclosed herein which fall within the meaning and scope of the claims are to be embraced thereby.

All references, including publications, patents, and patent applications, cited herein are hereby incorporated by reference to the same extent as if each reference were individually and specifically indicated to be incorporated by reference and were set forth in its entirety herein. The references discussed herein are provided solely for their disclosure prior to the filing date of the present application. Nothing herein is to be construed as an admission that the inventors are not entitled to antedate such disclosure by virtue of prior invention.

What is claimed is:

1. A detection-enhancement element for a biological assay, comprising:
a substrate;
a metallic layer on at least one surface of the substrate and including at least one nanocavity;
a transparent film positioned between the substrate and the metallic layer; and
capture molecules within the at least one nanocavity.

2. The detection-enhancement element of claim 1, wherein a surface of the metallic layer is passivated to prevent specific and non-specific binding of the capture molecules to the metallic layer outside of the at least one nanocavity.

3. The detection-enhancement element of claim 1, wherein the metallic layer comprises a material selected from the group consisting of gold, silver, aluminum, and combinations thereof.

4. The detection-enhancement element of claim 1, wherein the transparent film includes a material selected from the group consisting of titanium, chromium, and oxides and combinations thereof.

5. The detection-enhancement element of claim 1, wherein the thickness of the transparent film is between 2 to 15 nanometers.

6. The detection-enhancement element of claim 1, wherein the transparent film has a thickness and the at least one nanocavity has a lateral dimension, and the thickness and the lateral dimension have a ratio in the range of 1:4 to 1:100.

7. The detection-enhancement element of claim 6, wherein the thickness and the lateral dimension have a ratio in the range of 1:4 to 1:40.

8. The detection-enhancement element of claim 1, wherein the metallic layer has a thickness between 50 and 200 nanometers.

9. The detection-enhancement element of claim 1, wherein the metallic layer has a thickness between about 75 and 125 nanometers.

10. The detection-enhancement element of claim 1, wherein the metallic layer has a thickness less than 75 nanometers.

11. The detection-enhancement element of claim 1, wherein the at least one nanocavity has a lateral dimension between 65 and 190 nanometers.

12. The detection-enhancement element of claim 1, wherein the at least one nanocavity has a lateral dimension between about 100 to 140 nanometers.

13. The detection-enhancement element of claim 1, wherein the at least one nanocavity has a lateral dimension between about 65 to 85 nanometers.

14. The detection-enhancement element of claim 1, wherein the at least one nanocavity has a lateral dimension between about 120 to 160 nanometers.

15. The detection-enhancement element of claim 1, wherein the at least one nanocavity has a lateral dimension between about 150 to 190 nanometers.

16. The detection-enhancement element of claim 1, wherein the thickness of the transparent film and the thickness of the metallic layer have a ratio of about 1:5 to 1:30.

17. The detection-enhancement element of claim 1, wherein the at least one nanocavity is an array of multiple nano cavities.

18. The detection-enhancement element of claim 1, wherein the at least one nanocavity comprises a tapered sidewall with an angle, wherein the angle of the tapered sidewall with respect to a surface parallel to the substrate is sufficiently different than 90° to provide an enhancement of the transmission of light through the at least one cavity, an enhancement of the intensity of light within the at least one cavity, or both, that is greater than the enhancement if the angle was 90°.

19. The detection-enhancement element of claim 1, further comprising at least one change in a sidewall within the at least one cavity including a change in angle, a change in material, a change in width, or combinations thereof sufficient to provide an enhancement of the transmission of light through the at least one cavity, an enhancement of the intensity of light within the at least one nanocavity, or both, that is greater than the enhancement without the change in the sidewall.

20. The detection-enhancement element of claim 1, wherein the at least one cavity extends through the transparent film to a top surface of the substrate.

21. The detection-enhancement element of claim 1, wherein the at least one cavity has a cavity diameter and a cavity shape adapted to enhance transmission of light through the at least one nanocavity.

22. The detection-enhancement element of claim 1, wherein the transparent film causes an improvement in light transmission by a factor of at least 3.
